(19) United States
(12) Patent Application Publication
(10) Pub. No.: US 2024/0011976 A1
(43) Pub. Date: Jan. 11, 2024

(54) ADDITIVES FOR REDUCING NON-SPECIFIC INTERACTIONS BETWEEN FLUORESCENT POLYMER CONJUGATES AND CELLS IN A BIOLOGICAL SAMPLE

(71) Applicant: Beckman Coulter, Inc., Brea, CA (US)

(72) Inventors: Rajesh Venkatesh, Bangalore (IN); Shiva Ranjini Srinivasan, Bangalore (IN); Valerie Mallet Designe, Marseille (FR); Sylvain Monseaux, Marseille (FR); Geraldine Biechele, Marseille (FR)

(21) Appl. No.: 18/036,346

(22) PCT Filed: Nov. 12, 2021
(86) PCT No.: PCT/US2021/059254

§ 371 Date: May 10, 2023

Related U.S. Application Data
(60) Provisional application No. 61/113,703, filed on Nov. 12, 2008.

(51) Int. Cl. G01N 33/53; G01N 33/569; C09B 69/10; C09B 67/20; C08G 61/10

(52) U.S. Cl. CPC G01N 33/5306; G01N 33/56972; C09B 69/101; C09B 67/0065; C08G 61/10; C08G 2261/11; C08G 2261/122; C08G 2261/3142; C08G 2261/145; C08G 2261/1646; C08G 2261/72

(57) ABSTRACT

The disclosure relates to methods and compositions for reducing or eliminating non-specific binding of at least one dye conjugate to cells in a biological sample. A dye conjugate is contacted with at least one zwitterionic or anionic surfactant before, during or after the dye conjugate is contacted with a blood sample, resulting in substantially reduced non-specific binding of the dye conjugate to cells in the biological sample.

This application is being filed 12 Nov. 2021, as a PCT International Patent application and claims the benefit of priority to U.S. Provisional Application Ser. No. 63/113,703, filed 13 Nov. 2020, which is incorporated by reference herein in its entirety.

### BACKGROUND

Polymer dye conjugates are bright and provide excellent performance that can be utilized in single color or multi-color flow cytometry assays. In general, polymer dye conjugates exhibit high brightness due to their unique and complex structure. But that same unique and complex structure also may lead to some significant limitations. The instant disclosure addresses these limitations.

### SUMMARY

Because of their nature, polymer dye conjugates can bind non-specifically to cells in a biological sample, such as monocytes and granulocytes in a peripheral blood sample. Non-specific binding could lead to misinterpretation, resulting in false positive inferences. For example, when a polymer dye conjugate comes in contact with blood during the analysis of cellular markers, the conjugates may bind to cells, such as monocytes and/or granulocytes, non-specifically thereby either giving a signal that can be misinterpreted as positive population or pulls out populations other than the desired ones.

The instant disclosure provides a solution to these and other problems associated with use of polymer dye conjugates. In some embodiments, the disclosure provides a composition for reducing or eliminating non-specific binding of a dye conjugate to cells in a biological sample, the composition comprising a dye conjugate and a surfactant as described herein.

In some embodiments, the instant disclosure provides a method for reducing or eliminating non-specific binding of at least one dye conjugate to cells in biological sample, the method comprising: contacting that at least one dye conjugate with at least one zwitterionic surfactant before, during, or after the dye conjugate is contacted with the biological sample, the contacting resulting in decreased non-specific binding of the at least one dye conjugate in the sample. In some embodiments, the instant disclosure provides a method for reducing or eliminating non-specific binding of at least one to cells in a blood sample, the method comprising: contacting the at least one dye conjugate with at least one anionic surfactant before, during or after the dye conjugate is contacted with a blood sample, the contacting resulting in decreased non-specific binding of the at least one dye conjugate in the blood sample. The compositions and methods of the disclosure reduce or eliminate non-specific binding of a polymer dye conjugate or a non-polymeric dye conjugate to monocytes and/or granulocytes in a blood sample.

A method is provided for reducing or eliminating non-specific binding of at least one dye conjugate in a biological sample, such as a blood sample, the method comprising: contacting the at least one dye conjugate with at least one zwitterionic or anionic surfactant before, during, or after the polymer dye conjugate is contacted with a biological sample, the contacting resulting in decreased non-specific binding of the at least one polymer dye conjugate in the biological sample.

In some embodiments, the biological sample may be a blood sample. In some embodiments, the cell may be white blood cell(s) and the decreased non-specific binding may comprise decreased non-specific binding to a white blood cell in the blood sample. In some embodiments, the white blood cell is selected from the group consisting of monocytes and granulocytes.

In some embodiments, the method comprises adding the surfactant to the polymer dye conjugate before contacting the polymer dye conjugate with the biological sample, such as a peripheral blood sample.

In some embodiments, the method comprises adding the surfactant to the blood sample before the contacting with the polymer dye conjugate.

The surfactant may be a compound of the formula:

R^{1′}[CO—X(CH₂)ⱼ]_{g}—[N⁺(R^{2′})(R^{3′})]ₖ—(CH₂)_{f}—[CH(OH)CH₂]ₕ—Y⁻, wherein
R^{1′} is a saturated or unsaturated C₅₋₂₄ alkyl;
X is NH, NR^{4′}, wherein R^{4′} is C₁₋₄ alkyl, O or S;
j is an integer from 1 to 10;
g is 0 or 1;
R^{2′} and R^{3′} are independently a C₁₋₄ alkyl;
k is 0 or 1;
the hydroxyl is optionally substituted by methyl, ethyl, hydroxymethyl, or hydroxyethyl;
f is an integer from 0 to 4;
h is 0 or 1; and
Y is COO, SO₃, OPO(OR^{5′})O, or P(O)(OR^{5′})O, wherein R^{5′} is H or C₁₋₄ alkyl, and when k=0, the surfactant may be in acidic form, or sodium, or potassium salts thereof.

In some embodiments, the surfactant may be a zwitterionic surfactant compound of the formula:

R^{1′}[CO—X(CH₂)ⱼ]_{g}—N⁺(R^{2′})(R^{3′})—(CH₂)_{f}—[CH(OH)CH₂]ₕ—Y⁻,
wherein:
R^{1′} is saturated or unsaturated C₅₋₂₄ alkyl;
X is NH or NR^{4′}, wherein R^{4′} is C₁₋₄ alkyl, O or S;
j is an integer from 1 to 10;
g is 0 or 1;
R^{2′} and R^{3′} are independently a C₁₋₄ alkyl;
the hydroxyl is optionally substituted by methyl, ethyl, hydroxymethyl, or hydroxyethyl;
f is an integer from 1 to 4;
h is 0 or 1; and
Y is COO, SO₃, OPO(OR^{5′})O or P(O)(OR^{5′})O, wherein R^{5′} is H or a C₁₋₄ alkyl residue.

The zwitterionic surfactant may be a compound of the formula:

R^{1′}—N⁺(CH₃)₂—CH₂COO⁻;

R^{1′}—CO—NH(CH₂)₃—N⁺(CH₃)₂—CH₂COO⁻;

R^{1′}—N⁺(CH₃)₂—CH₂CH(OH)CH₂SO₃⁻; or

R^{1′}—CO—NH—(CH₂)₃—N⁺(CH₃)₂—CH₂CH(OH)CH₂SO₃⁻.

In some embodiments, the zwitterionic surfactant may be selected from the group consisting of almondamidopropyl betaine, apricotamidopropyl betaine, avocadamidopropyl betaine, babassuamidopropyl betaine, behenamidopropyl betaine, behenyl betaine, canolamidopropyl betaine, capryl/capramidopropyl betaine, camitine, cetyl betaine, cocamidoethyl betaine, cocamidopropyl betaine, cocamidopropyl hydroxysultaine, coco betaine, coco hydroxysultaine, coco/oleamidopropyl betaine, coco sultaine, decyl betaine, dihydroxyethyl oleyl glycinate, dihydroxyethyl soy glycinate, dihydroxyethyl stearyl glycinate, dihydroxyethyl tallow glycinate, dimethicone propyl PG-betaine, drucamidopropyl hydroxysultaine, hydrogenated tallow betaine, isostearamidopropyl betaine, lauramidopropyl betaine, lauryl betaine, lauryl hydroxysultaine, lauryl sultaine, milk amidopropyl betaine, milkamidopropyl betaine, myristamidopropyl betaine, myristyl betaine, oleamidopropyl betaine, oleamidopropyl hydroxysultaine, oleyl betaine, olivamidopropyl betaine, palmamidopropyl betaine, palmitamidopropyl betaine, palmitoyl camitine, palm kernel amidopropyl betaine, polytetrafluoroethylene acetoxypropyl betaine, ricinoleamidopropyl betaine, sesamidopropyl betaine, soyamidopropyl betaine, stearamidopropyl betaine, stearyl betaine, tallowamidopropyl betaine, tallowamidopropyl hydroxysultaine, tallow betaine, tallow dihydroxyethyl betaine, undecylenamidopropyl betaine, and wheat germ amidopropyl betaine. In some embodiments, the surfactant is lauryl betaine.

In some embodiments, the surfactant may be an anionic surfactant compound of the formula:

R^{1′}[CO—X(CH₂)ⱼ]_{g}—(CH₂)_{f}—[CH(OH)CH₂]ₕ—Y⁻, wherein
R^{1′} is a saturated or unsaturated C₅₋₂₄ alkyl;
X is NH, NR^{4′}, wherein R^{4′} is C₁₋₄ alkyl, O, or S;
j is an integer from 1 to 10;
g is 0 or 1;
R^{2′} and R^{3′} are independently a C₁₋₄ alkyl;
the hydroxyl is optionally substituted by methyl, ethyl, hydroxymethyl, or hydroxyethyl;
f is an integer from 0 to 4;
h is 0 or 1; and
Y is COO, SO₃, OPO(OR^{5′})O or P(O)(OR^{5′})O, wherein R^{5′} is H or C₁₋₄ alkyl and wherein the anionic surfactant may be in acidic form, or sodium, or potassium salt forms thereof. In some embodiments, f=0. In some embodiments, f=1. In some embodiments, f=3. In some embodiments, f=4. In some embodiments, Y is COO or SO₃. In some embodiments, R^{2′} and R^{3′} are methyl.

The anionic surfactant may be a compound according to the formula

R^{1′}—CO—N(CH₃)—CH₂COO⁻; or

R^{1′}—CO—N(CH₃)—CH₂—SO₃—, and sodium or potassium salts thereof, wherein
R^{1′} is saturated or unsaturated C₅₋₂₄ alkyl. In some embodiments, R^{1′} may be a saturated or unsaturated C₇₋₁₉ alkyl, or C₁₁₋₁₇ alkyl.

In some embodiments, the anionic surfactant may be selected from the group consisting of N-lauroyl sarcosine, sodium lauroylsarcosinate, sodium palmitoyl sarcosinate, sodium stearoyl sarcosinate, N-methyl-N-(1-oxotetradecyl)-glycine sodium salt, sodium caproyl sarcosinate, sodium capryloyl sarcosinate, N-methyl-N-(1-oxo-9-octadecen-1-yl)-glycine, sodium salt, sodium oleoyl sarcosinate, and sodium linoleoyl sarcosinate. In some embodiments, the anionic surfactant is N-lauroyl sarcosine.

In some embodiments, the polymer dye conjugate comprises a binding partner conjugated to a polymer dye having the structure of Formula III:

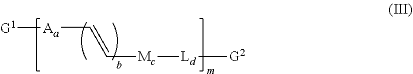

wherein,
   each A is independently selected from the group consisting of an aromatic co-monomer and a heteroaromatic co-monomer;
   each optional M is independently selected from the group consisting of an aromatic co-monomer, a heteroaromatic co-monomer, a bandgap-modifying monomer, optionally substituted ethylene, and ethynylene;
   each optional L is a linker moiety;
   each G¹ and G² are independently selected from an unmodified polymer terminus and a modified polymer terminus;
   a, c, and d define the mol % of each unit which each can be evenly or randomly repeated and where each a is a mol % from 10 to 100%, each c is a mol % from 0 to 90%, and each d is a mol % from 0 to 25%;
   each b is independently 0 or 1;
   and each m is an integer from 1 to about 10,000.

In some embodiments A comprises a DHP moiety. In some embodiments, A comprises a fluorene moiety. In some embodiments, A comprises a DHP and a fluorene moiety.

In some embodiments, the polymer dye conjugate is a polymer of Formula I:

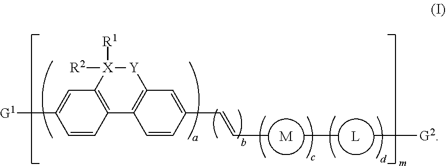

wherein
each X is independently C or Si;
each Y is independently CR¹R² or SiR¹R²;
each R¹ is independently an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a water solubilizing moiety:

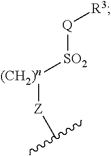

each R² is independently H, alkyl, alkenyl, alkynyl, cycloalkyl, haloalkyl, alkoxy, (hetero)aryloxy, aryl, (hetero)arylamino, a PEG group, an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a water solubilizing moiety

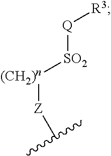

each R³ is independently selected from the group consisting of H, alkyl, alkene, alkyne, cycloalkyl, haloalkyl, alkoxy, (hetero)aryloxy, aryl, (hetero)arylamino, and a PEG group; each Z is independently selected from the group consisting of C, O, and N; each Q is independently selected from the group consisting of a bond, NH, NR⁴, and CH₂; and
each subscript n is independently an integer from 0 to 20;
each M is a unit capable of altering the polymer band gap and are evenly or randomly distributed along the polymer main chain; L is a linker; G¹ and G², which are each independently selected from the group consisting of hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted dihydrophenanthrene (DHP), optionally substituted fluorene, aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof conjugated to a binding partner. In some embodiments, L is an aryl or heteroaryl group evenly or randomly distributed along the polymer main chain and substituted with one or more pendant chains terminated with a functional group selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof conjugated to a binding partner; a, c, and d, define the mol % of each unit within the structure which each can be evenly or randomly repeated and where a is a mol % from 10 to 100%, c is a mol % from 0 to 90%, and each d is a mol % from 0 to 25%; each b is independently 0 or 1; m is an integer from 1 to about 10,000; and each n is independently an integer from 1 to 20.

The binding partner may be a molecule or complex of molecules capable of specifically binding to target analyte. The binding partner may be a protein, an affinity ligand, an antibody, or an antibody fragment. In some embodiments, the binding partner may be selected from the group consisting of a monoclonal antibody, a polyclonal antibody, an immunoglobulin, an immunologically active portion of an immunoglobulin, a single chain antibody, Fab fragment, Fab′ fragment, and F(ab′)2 fragments, and scFv fragment.

A composition is provided comprising a polymer dye conjugate; an aqueous buffer; and a zwitterionic or anionic surfactant. The composition may comprise the zwitterionic or anionic surfactant at a concentration below the critical micellar concentration (CMC). In some embodiments, the surfactant may be at a concentration of 0.05 to 0.25% (w/v), 0.06 to 0.20% (w/v), or 0.08 to 0.16% (w/v). The aqueous buffer may comprise an additional additive selected from the group consisting of a protein stabilizer, a preservative, and an additional surfactant. The composition may exhibit, following exposure to a blood sample and flow cytometry analysis, decreased non-specific binding of polymer dye conjugate to white blood cells in a sample. The decreased non-specific binding may be compared to the same composition without the zwitterionic or anionic surfactant. The white blood cells may be selected from the group consisting of monocytes and granulocytes.

### BRIEF DESCRIPTION OF THE DRAWINGS

FIG. 1A shows a plot of fluorescence intensity as a function of wavelength for fluorene (FF), dihydrophenanthrene (DD) and fluorene-DHP (DF) polymer dyes.

FIG. 1B shows a graph of absorption spectra of fluorene (FI-FI) polymer and dihydrophenanthrene (DHP-DHP) polymer. The DHP-DHP polymer (black curve) exhibits lambda max (λmax) at 390 and 410 nm, whereas the FI-FI (grey curve) polymer shows lambda max (λmax) at about 400 nm.

FIG. 2 shows flow cytometry dot plots of unstained blood cells (upper panel); blood cells stained with polymer dye and no surfactant (lower left panel); and blood cells stained with a composition comprising polymer dye and a surfactant (lower right). The fluorescent polymer dye SN v605 without antibody was used to stain a blood sample and analyzed in a flow cytometer. Polymer dye without surfactant exhibited non-specific binding to the monocytes/granulocytes (lower left). Polymer dye with EMPIGEN BB® exhibited a substantial decrease in the non-specific binding to monocytes/granulocytes (lower right).

FIG. 3 shows dot plots for blood cells without polymer dye conjugate (upper panel) and blood cells stained with SN 605-CD20 conjugate with (lower left panel) and without surfactant (lower right panel). The percentage of non-specifically bound granulocytes was reduced (check the “P2” gate in the dot plot) with the usage of surfactant. Also, the functional aspect of the conjugate did not change (check the “P1” gate in the dot plot), since the percentage of the positive population is similar in both the cases.

FIG. 4 shows a bar graph of Median Fluorescence Intensity (MdFI) values of monocytes in presence and absence of surfactant for two lots of polymer dye conjugates (SN v605-CD20) compared to unstained monocytes (autofluorescence). In the presence of surfactant, the non-specific interaction on monocytes was substantially reduced for both Lot-1 and Lot-2 SN605 CD20 conjugates.

FIG. 5 shows a bar graph of MdFI values of granulocytes in presence and absence of surfactant for two lots of polymer dye conjugates (SN v605-CD20) compared to unstained granulocytes (autofluorescence). In the presence of surfactant, the non-specific interaction on granulocytes was reduced for both Lot-1 and Lot-2 SN605 CD20 conjugates.

FIG. 6 shows dot plots of blood cells without polymer dye conjugate (upper left) and stained with SN v786-CD103 conjugate with Empigen BB® surfactant (lower left) and without surfactant (upper right). The dot plot compares one of the claimed polymers and BV786-CD103, tandem fluorochrome (lower right) (available from Becton Dickinson). The percentage of non-specifically bound granulocytes and monocytes were reduced (check the “P1” and “P2” gate respectively in the dot plot) in the presence of surfactant.

FIG. 7 shows a bar graph of MdFI values of monocytes in presence and absence of surfactant for two lots of polymer dye conjugates (SN v786-CD103) compared to unstained monocytes (autofluorescence). In the presence of surfactant, non-specific binding of polymer conjugates to monocytes was substantially reduced for both lots of the polymer dye conjugates.

FIG. 8 shows a bar graph of MdFI values of granulocytes in presence and absence of surfactant for two lots of polymer conjugates compared to unstained granulocytes (autofluorescence). In the presence of surfactant, non-specific binding of polymer conjugates to granulocytes was substantially reduced for both lots of the polymer dye conjugates.

FIG. 9 shows dot plots of blood cells stained with SN v605-CD20 conjugate with and without surfactants (upper panel), where the lower left panel is nonionic surfactant Tween-20 and the lower right panel is nonionic surfactant Pluronic F-68. The percentage of non-specifically bound monocytes was not reduced (check the “non-specific monocytes” gate in the dot plot) with the usage of nonionic surfactants Tween-20 and Pluronic F-68.

FIG. 10 shows a dot plot of blood cells without dye conjugate (upper left panel); blood cells stained with SN v605-CD20 conjugate with BSA (upper right panel), oxidized BSA (lower left panel), and BSA-Cy5-ox (lower right panel). The percentage of non-specifically bound monocytes and granulocytes was not substantially reduced (check the “P1” gate in the dot plot) with the usage of protein blockers.

FIG. 11 shows three graphs each showing the effect of surfactant concentration on negative monocytes (MFI) for Donor 1 (D1) and Donor 2 (D2) blood samples for unstained and stained samples for SN v428 CD19 (FIG. 11, upper panel), SN v428 CD22 (FIG. 11, lower panel), and SN v428 CD25 (FIG. 11, middle panel) specificities. SN conjugates in the presence of 0.06 to 0.20% Empigen BB® exhibited lower non-specific monocyte interactions than in absence of surfactant.

FIG. 11 (cont.) shows three graphs each showing the effect of surfactant concentration on negative monocytes (MFI) for Donor 1 (D1) and Donor 2 (D2) blood samples for unstained and stained samples for SN v428 CD19 (FIG. 11, upper panel), SN v428 CD22 (FIG. 11, lower panel), and SN v428 CD25 (FIG. 11, middle panel), with data shown as negative monocyte MFI in % of no Empigen BB® samples. Samples stained with BD polymer dye conjugates in the presence of 0.06 to 0.20% Empigen BB® exhibited lower percent of non-specific monocyte interactions than in absence of surfactant.

FIG. 12 (nine graphs) shows the effect of surfactant concentration on negative granulocytes (upper three panels), positive lymphocytes (middle panels), and positive lymphocytes in % of no Empigen BB® samples (lower three panels). Unstained and stained blood samples from donor 1 (D1) and donor 2 (D2) are shown. Somewhat lower negative interaction to granulocytes was exhibited in presence of 0.06 to 0.20% surfactant, compared to absence of surfactant for each of CD19 BD, CD25 BD, and CD22 BD polymer dye conjugates (upper three panels). Positive lymphocyte data were similar or slightly higher in presence of surfactant, compared to absence of surfactant for each of CD19 BD, CD25 BD, and CD22 BD polymer dye conjugates (middle three and lower three panels).

FIG. 13 shows dot plots of SS/FL9 staining patterns for SN v428 CD19 Lot #D19-094 polymer dye conjugate at 0.5 μg/test without surfactant, with 0.06%, 0.12%, and 0.2% Empigen BB® surfactant, and CD19 BV-421 conjugate (Becton Dickinson) at its commercial dose on donor 1 (upper panels) and donor 2 (lower panels) blood samples.

FIG. 14 shows dot plots of SS/FL9 staining patterns for SN v428 CD25 Lot #D19-107 polymer dye conjugate at 0.5 μg/test without surfactant, with 0.06%, 0.12%, and 0.2% surfactant and CD25 BV-421 conjugate (Becton Dickinson) at its commercial dose on donor 1 (upper panels) and donor 2 (lower panels) blood samples.

FIG. 15 shows dot plots for SN v428 CD22 Lot #D19-109 polymer dye conjugate at 0.5 μg/test without surfactant, with 0.06%, 0.12%, and 0.2% surfactant and CD22 BV-421 conjugate (Becton Dickinson) at its commercial dose on donor 1 (upper panels) and donor 2 (lower panels) blood samples.

FIG. 16 shows dot plots with the percentage of dead cells at up to 0.2% surfactant. CD19-SNv428 D19-094 without EMPIGEN BB® (negative control) and with 0.06%, 0.12%, and 0.2% EMPIGEN BB®, was tested on donor 1 and donor 4 whole blood samples, stained with 7-ADD, to evaluate the percentage of dead cells in each condition. Whole blood samples that had been preserved for greater than 24 hours were added as positive control of 7-AAD staining (left panel, 12% dead cells). The percentage of dead cells was not substantially increased by the presence of EMPIGEN BB® at up to 0.2%, when compared to samples without surfactant.

FIG. 17A shows a dot plot of a peripheral blood sample without single color conjugate, evident as there is no population in the CD20+ gate.

FIG. 17B shows a positive control dot plot of a peripheral blood sample in the presence of CD20-SN v605 single-color conjugate in a buffer composition containing BSA, sodium azide, Pluronic™ F-68 (PF-68) and Empigen BB® as additives. When compared to negative control dot plot (FIG. 17C), the % population in the gate “Mons Non-specific binding” and “Grans Non-specific binding are each considerably reduced, indicating the effectiveness of Empigen BB® in eliminating or reducing non-specific binding to monocytes and granulocytes.

FIG. 17C shows a negative control dot plot of a peripheral blood sample in the presence of CD20-SN v605 single-color conjugate in a buffer composition containing only BSA, PF-68 and sodium azide as additives.

FIG. 17D shows a test dot plot of a peripheral blood sample in presence of CD20-SN v605 single-color conjugate in a buffer composition containing BSA, sodium azide, PF-68 and NLS (0.16% w/v) as additives.

FIG. 17E shows a test dot plot of a peripheral blood sample in presence of CD20-SN v605 single-color conjugate in a buffer composition containing BSA, sodium azide, PF-68 and NLS (0.08% w/v) as additives.

### DETAILED DESCRIPTION OF THE DISCLOSURE

Reference will now be made in detail to certain embodiments of the disclosed subject matter, examples of which are illustrated in part in the accompanying drawings and Examples. While the disclosed subject matter will be described in conjunction with the enumerated claims, it will be understood that the exemplified subject matter is not intended to limit the claims to the disclosed subject matter.

### General Disclosure

The disclosure generally relates to compositions, and methods for detecting analytes in a sample using compositions comprising at least one surfactant and at least one polymer dye conjugated to binding partners (e.g., antibodies), for example a fluorescent polymer dye conjugated to binding partner. More specifically, the disclosure relates to a method for reducing or eliminating non-specific binding of at least one polymer dye conjugate in a biological sample, such as a blood sample, the method comprising: contacting the at least one polymer dye conjugate with at least one zwitterionic or anionic surfactant before, during, or after the polymer dye conjugate is contacted with a biological sample, such as a blood sample, the contacting resulting in decreased non-specific binding of the at least one polymer dye conjugate to cells, such as white blood cells in the blood sample. The surfactant can be added to the blood sample before the contacting. The surfactant can be added to the polymer dye conjugate prior to contacting with a biological sample.

### Definitions

The abbreviations used herein have their conventional meaning within the chemical and biological arts.

The singular forms “a”, “an” and “the” are intended to include the plural forms as well, unless the context clearly indicates otherwise.

The term “and/or” refers to and encompasses any and all possible combinations of one or more of the associated listed items. Unless otherwise specified, the term phrase “room temperature” refers to 18 to 27° C.

Unless otherwise specified, the term “percent”, or “%” refers to weight percent.

All patents, patent applications and publications referred to herein are incorporated by reference in their entirety.

The term “Analyte” refers to a molecule, compound, or other component in a sample. Analytes may include but are not limited to peptides, proteins, polynucleotides, organic molecules, sugars and other carbohydrates, and lipids.

The term “Binding partner” refers to a molecule capable of specifically binding an analyte. A binding partner can be any of a number of different types of molecules, including an antibody or antigen-binding fragment thereof, or other protein, peptide, polysaccharide, lipid, a nucleic acid or nucleic-acid analog, such as an oligonucleotide, aptamer, or PNA (peptide nucleic acids).

The term “CD” refers to Cluster of differentiation.

The term “Compensation” in flow cytometry is a mathematical process of correcting for fluorescence spillover (spectral overlap of multiparameter flow cytometric data). For example, compensation may be performed by removing the signal of any given fluorochrome from all detectors except the one devoted to measuring that dye. Since fluorochromes may have wide-ranging spectrum, they can overlap, causing the undesirable confusion during data analysis.

The term “Labeled binding partner” refers to a binding partner that is conjugated to a dye. The term “Reactant solution” refers to solution comprising the labeled binding partner. In some embodiments, besides the labeled binding partner, a reactant solution may further comprise stabilizers, salt, buffer, surfactants, and/or other reagents.

The term “linker” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some embodiments, the linker is a branching linker that refers to a linking moiety that connects three or more groups. In certain cases, one, two, three, four or five or more carbon atoms of a linker backbone may be optionally substituted with a sulfur, nitrogen or oxygen heteroatom. In some embodiments, the linker backbone includes a linking functional group, such as an ether, thioether, amino, amide, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol, ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

A linker moiety can be attached to “A”, as taught in US Published Application No. 2020/0190253A1, which is incorporated herein by reference in its entirety, or to “L”, as taught in US Published Application No. 2019/0144601, which is incorporated here by reference in its entirety. A linker moiety can comprise a sulfonamide, disulfonamide, a selenomide, a sulfinamide, a sultam, a disulfinamide, an amide, a seleninamide, a phosphonamide, a phosphinamide, a phosphonamidate, or a secondary amine.

As described therein, and as each pertains to a linker moiety, the term “sulfonamide,” refers to a moiety-S(O)₂NR—; the term “disulfonamide,” refers to a moiety —S(O)₂NRS(O)₂—; the term “selenonamide,” refers to a moiety —Se(O)₂NR—; the term “sulfinamide,” refers to a moiety —S(O)NR—; the term “disulfinamide,” refers to a moiety —S(O)NRS(O)—; the term “seleninamide,” refers to a moiety —Se(O)NR—; the term “phosphonamide,” refers to a moiety —NR—PR(O)NR—; the term “phosphinamide,” refers to a moiety —PR(O)NR—; and the term “phosphonamidate,” refers to a moiety —O—PR(O)NR—; and the term “sultam” refers to a cyclic sulfonamide (e.g., wherein the R group is bonded to the sulfur atom via an alkylene moiety); wherein for each term the R group is independently H, alkyl, haloalkyl, or aryl.

The term “terminus” as used herein refers to termini on the conjugated polymer chains that can include a functional group that provides for bioconjugation. In some cases, such functionality is referred to as an end linker. The terminus may be, for example, hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted tetrahydropyrene (THP), optionally substituted fluorene, optionally substituted dihydrophenanthrene (DHP), aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to a substrate, or a binding agent

The term “MdFI” or “MDFI” refers to Median fluorescence intensity.

The term “% recruitment” refers to number of gated cells of relevant population.

The term “Multiplexing” herein refers to an assay or other analytical method in which multiple analytes can be assayed simultaneously.

The term “PEG” refers to polyethylene glycol, or poly(ethylene glycol). The number after “PEG” refers to the average molecular weight, where Mw refers to weight average molecular weight, and Mn refers to number average molecular weight.

The term “PBS” refers to phosphate buffered saline which is an aqueous buffer which may contain sodium chloride, disodium hydrogen phosphate, potassium chloride, and potassium dihydrogen phosphate. For example, PBS may contain milliQ water or deionized water and 137 mM NaCl, 2.7 mM KCl, 10 mM Na₂HPO₄, 1.8 mM KH₂PO₄. The pH may be about pH 7.0-7.4. The PBS may or may not be preserved with an azide such as sodium azide. PBS is an isotonic solution.

The acronym “SSC” refers to side scatter.

The acronym “WBC” refers to white blood cells.

A “dye” is a moiety that provides a detectable signal, which can be attached to or incorporated into a binding partner, either directly or indirectly. A dye used in the disclosure can be colored, fluorescent, or luminescent, and is typically detected by detector in flow cytometer, e.g., PMT or APD. Fluorescent dyes can be monomeric or polymeric. The fluorescent dye may be a fluorescent polymer dye. Polymeric dyes are particularly useful for analysis of chemical and biological targets. They are highly responsive optical reporters and efficient light absorbers, by virtue of the multiple chromophores they comprise. Fluorescent polymer dyes appropriate for use in the present disclosure are described herein, for example, in US 2019/0144601 and US 2020/0190253. Examples of polymeric dyes include, but are not limited to, conjugated polymers having repeat units of chromophore, aggregates of conjugated molecules, luminescent dyes attached via side chains to saturated polymers, semiconductor quantum dots and dendritic structures. Polymeric and monomeric dyes disclosed in U.S. Pat. Nos. 7,214,489, 8,354,239, 8,575,303 can also be used for the present invention.

As used herein, the term “ammonium” refers to a cation having the formula NHR₃⁺ where each R group, independently, is hydrogen or a substituted or unsubstituted alkyl, aryl, aralkyl, or alkoxy group. Preferably, each of the R groups is hydrogen.

As used herein, “oligoether” is understood to mean an oligomer containing structural repeat units having an ether functionality. As used herein, an “oligomer” is understood to mean a molecule that contains one or more identifiable structural repeat units of the same or different formula.

The term “sulfonate functional group” or “sulfonate,” as used herein, refers to both the free sulfonate anion (—S(═O)₂O—) and salts thereof. Therefore, the term sulfonate encompasses sulfonate salts such as sodium, lithium, potassium and ammonium sulfonate.

The term “sulfonamido” as used herein refers to a group of formula —SO₂NR— where R is hydrogen, alkyl or aryl.

The term “alkyl” as used herein refers to a straight or branched, saturated, aliphatic radical having the number of carbon atoms indicated. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together.

The term “cycloalkyl” as used herein refers to a saturated or partially unsaturated, monocyclic, fused bicyclic or bridged polycyclic ring assembly containing from 3 to 12 ring atoms, or the number of atoms indicated monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C₃₋₈cycloalkyl includes cyclopropyl, cyclobutyl, cyclopentyl, cyclohexyl, cyclooctyl, and norbornane.

The term “haloalkyl” as used herein refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, fluoromethyl, 1,2,3,4,5-pentafluoro-phenyl, etc. The term “perfluoro” defines a compound or radical which has at least two available hydrogens substituted with fluorine. For example, perfluorophenyl refers to 1,2,3,4,5-pentafluorophenyl, perfluoromethane refers to 1,1,1-trifluoromethyl, and perfluoromethoxy refers to 1,1,1-trifluoromethoxy.

As used herein, the term “halogen” refers to fluorine, chlorine, bromine and iodine.

The term “alkoxy” as used herein refers to an alkyl group, as defined above, having an oxygen atom that connects the alkyl group to the point of attachment. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

The term “alkene” as used herein refers to either a straight chain or branched hydrocarbon, having at least one double bond. Examples of alkene groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrenyl. The alkene group is typically monovalent, but can be divalent, such as when the alkenyl group links two moieties together.

The term “alkyne” as used herein refers to either a straight chain or branched hydrocarbon, having at least one triple bond. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. The alkynyl group is typically monovalent, but can be divalent, such as when the alkynyl group links two moieties together.

The term “aryl” as used herein refers to a monocyclic or fused bicyclic, tricyclic or greater, aromatic ring assembly containing 6 to 16 ring carbon atoms. For example, aryl may be phenyl, benzyl or naphthyl, preferably phenyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g. methylenedioxy or ethylenedioxy. Oxy-C₂-C₃-alkylene is also a divalent substituent attached to two adjacent carbon atoms of phenyl, e.g. oxyethylene or oxypropylene. An example for oxy-C₂-C₃-alkylene-phenyl is 2,3-dihydrobenzofuran-5-yl.

Preferred as aryl is naphthyl, phenyl or phenyl mono- or disubstituted by alkoxy, phenyl, halogen, alkyl or trifluoromethyl, especially phenyl or phenyl-mono- or disubstituted by alkoxy, halogen or trifluoromethyl, and in particular phenyl.

The term “aryloxy” as used herein refers to a O-aryl group, wherein aryl is as defined above. An aryloxy group can be unsubstituted or substituted with one or two suitable substituents. The term “phenoxy” refers to an aryloxy group wherein the aryl moiety is a phenyl ring. The term “heteroaryloxy” as used herein means an —O— heteroaryl group, wherein heteroaryl is as defined below. The term “(hetero)aryloxy” is use to indicate the moiety is either an aryloxy or heteroaryloxy group.

The terms “Polyethylene glycol” or “PEG” as used herein refer to the family of biocompatible water-solubilizing linear polymers based on the ethylene glycol monomer unit described by the formula —(CH2-CH2-O—)ₙ— or a derivative thereof. In some embodiments, “n” is 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, amino, acyl, carboxylic acid, carboxylate ester, acyloxy, and amido terminal and/or substituent groups.

The term “heteroaryl” as used herein refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl.

Preferably, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)r, or —S(O)₂NR′—. The substituent R′ in —NR′— and —S(O)₂NR′— is selected from hydrogen or unsubstituted (C₁-C₆)alkyl.

The term “(hetero)arylamino” as used herein refers an amine radical substituted with an aryl group (e.g., —NH-aryl). An arylamino may also be an aryl radical substituted with an amine group (e.g., -aryl-NH₂). Arylaminos may be substituted or unsubstituted.

The term “amine” as used herein refers to an alkyl groups as defined within, having one or more amino groups. The amino groups can be primary, secondary or tertiary. The alkyl amine can be further substituted with a hydroxy group. Amines useful in the present invention include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present invention.

The term “carbamate” as used herein refers to the functional group having the structure —NR″CO₂R′, where R′ and R″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. Examples of carbamates include t-Boc, Fmoc, benzyloxy-carbonyl, alloc, methyl carbamate, ethyl carbamate, 9-(2-sulfo)fluorenylmethyl carbamate, 9-(2,7-dibromo)fluorenylmethyl carbamate, Tbfmoc, Climoc, Bimoc, DBD-Tmoc, Bsmoc, Troc, Teoc, 2-phenylethyl carbamate, Adpoc, 2-chloroethyl carbamate, 1,1-dimethyl-2-haloethyl carbamate, DB-t-BOC, TCBOC, Bpoc, t-Bumeoc, Pyoc, Bnpeoc, V-(2-pivaloylamino)-1,1-dimethylethyl carbamate, NpSSPeoc.

The term “carboxylate” as used herein refers to the conjugate base of a carboxylic acid, which generally can be represented by the formula RCOO. For example, the term “magnesium carboxylate” refers to the magnesium salt of the carboxylic acid.

The term “activated ester” as used herein refers to carboxyl-activating groups employed in peptide chemistry to promote facile condensation of a carboxyl group with a free amino group of an amino acid derivative. Descriptions of these carboxyl-activating groups are found in general textbooks of peptide chemistry; for example K. D. Kopple, “Peptides and Amino Acids”, W. A. Benjamin, Inc., New York, 1966, pp. 50-51 and E. Schroder and K. Lubke, “The Peptides”; Vol. 1, Academic Press, New York, 1965, pp. 77-128.

The terms “hydrazine” and “hydrazide” refer to compounds that contain singly bonded nitrogens, one of which is a primary amine functional group.

The term “aldehyde” as used herein refers to a chemical compound that has an —CHO group.

The term “thiol” as used herein refers to a compound that contains the functional group composed of a sulfur-hydrogen bond. The general chemical structure of the thiol functional group is R—SH, where R represents an alkyl, alkene, aryl, or other carbon-containing group of atoms.

The term “silyl” as used herein refers to Si(R^{z})₃ wherein each R^{z} independently is alkyl aryl or other carbon-containing group of atoms.

The term “diazonium salt” as used herein refers to a group of organic compounds with a structure of R—N₂⁺X⁻, wherein R can be any organic residue (e.g., alkyl or aryl) and X is an inorganic or organic anion (e.g., halogen).

The term “triflate” also referred to as trifluoromethanesulfonate, is a group with the formula CF₃SO₃.

The term “boronic acid” as used herein refers to a structure —B(OH)₂. It is recognized by those skilled in the art that a boronic acid may be present as a boronate ester at various stages in the synthesis of the quenchers. Boronic acid is meant to include such esters. The term “boronic ester” or “boronate ester” as used herein refers to a chemical compound containing a —B(Z¹)(Z²) moiety, wherein Z¹ and Z² together form a moiety where the atom attached to boron in each case is an oxygen atom. The boronic ester moiety can be a 5-membered ring. The boronic ester moiety can be a 6-membered ring. The boronic ester moiety can be a mixture of a 5-membered ring and a 6-membered ring.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

As used herein, “dye conjugate” refers to a binding partner conjugated to a non-polymeric or polymeric dye.

As used herein, “SN” refers to “Super Nova” dyes commercially available from Beckman Coulter, Inc.

As used here “EMPIGEN BB®” refers to a zwitterionic surfactant (CAS Number 66455-29-6) comprising N,N-dimethyl-N-dodecylglycine betaine at a concentration of ˜30% betaine in aqueous solution.

The term “about” as used herein can allow for a degree of variability in a value or range, for example, within 10%, within 5%, or within 1% of a stated value or of a stated limit of a range.

As used herein, “specific binding” refers to binding of an antibody or other binding partner (e.g., in a polymer conjugate dye) to an epitope on a cell or target analyte to which the antibody or binding partner is targeted.

As used herein, “non-specific binding” refers to binding of an antibody or other binding partner (e.g., in a polymer conjugate dye) to a cell or sample component that does not comprise an epitope to which the antibody or other binding partner is targeted. For example, non-specific binding occurs when an antibody binds to a cell that does not have an epitope specifically for that antibody.

As used herein, “reducing” or “eliminating” of non-specific binding of the polymer dye conjugate can refer to when the “negatives” (e.g., negative granulocyte, monocyte, and lymphocyte populations) mean fluorescence intensity (MFI), in % relative to when no surfactant is used, is decreased by at least about 50% (e.g., by at least about 60%, at least about 70%, at least about 80%, at least about 90%, at least about 95%, at least 99% or more; from about 50% to about 95%, about 50% to about 75%, about 60% to about 80% or about 65% to about 90%). In other words, the % reduction of at least one of monocyte, granulocyte, and lymphocyte background staining, in % relative to when no surfactant is used, is decreased by at least about 50% (e.g., by at least about 60%, at least about 70%, at least about 80%, at least about 90%, at least about 95%, at least 99% or more; from about 50% to about 95%, about 50% to about 75%, about 60% to about 80% or about 65% to about 90%).

The term “substantial” or “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” or “substantially free of” as used herein refers to less than about 1%, 0.5%, 0.1%, 0.05%, 0.001%, or at less than about 0.0005% or less, about 0%, below quantitation limits, below detectable limits, or 0%.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In certain embodiments, a dye composition is provided comprising at least one polymer dye conjugate and at least one suitable zwitterionic surfactant.

In certain embodiments, a dye composition is provided comprising at least one polymer dye conjugate with at least one suitable anionic surfactant.

In some embodiments, a method is provided for reducing or eliminating non-specific binding of at least one polymer dye conjugate to a cell in a biological sample, such as a blood sample, comprising contacting at least one polymer dye conjugate with at least one zwitterionic and/or anionic surfactant before, during, and/or after the at least one polymer dye conjugate is contacted with the biological sample. The steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process. Accordingly, in some instances the at least one polymer dye conjugate can be contacted with at the least one zwitterionic or anionic surfactant before the at least one polymer dye conjugate is contacted with the blood sample. In some instances, the at least one polymer dye conjugate can be contacted with at the least one zwitterionic or anionic surfactant at the same time the at least one polymer dye conjugate is contacted with the blood sample.

### Surfactant

Various types of surfactants were explored for reducing or preventing non-specific interactions of the polymer dye conjugate with biological samples.

Suitable surfactants may be zwitterionic surfactants or certain anionic surfactants. Examples of suitable surfactants include surfactants of the general formula

R^{1′}[CO—X(CH₂)ⱼ]_{g}—[N⁺(R^{2′})(R^{3′})]ₖ—(CH₂)_{f}—[CH(OH)CH₂]ₕ—Y⁻, wherein R^{1′} is a saturated or unsaturated C₅₋₂₄ alkyl, such as a C₆₋₂₂, C₅₋₂₁, C₇₋₁₉, C₁₁₋₁₇, or C₈₋₁₈ alkyl, a saturated C₁₀₋₁₆ alkyl or a saturated C₁₂₋₁₄ alkyl; X is NH, NR^{4′}, wherein R^{4′} is C₁₋₄ alkyl, O or S; j is an integer from 1 to 10, such as from 2 to 5 and 3; g is 0 or 1, R^{2′} and R^{3′} are each, independently, a C₁₋₄ alkyl, such as ethyl or methyl; optionally hydroxy substituted by a hydroxyethyl group or a methyl; k is 0 or 1; f is an integer from 0 to 4, such as 0, 1, 2, 3, or 4; h is 0 or 1; and Y is COO, SO₃, OPO(OR^{5′})O or P(O)(OR^{5′})O, wherein R^{5′} is H or C₁₋₄ alkyl, and when k=0, the surfactant may be in acidic form, or sodium, or potassium salts thereof.

The surfactant can be present at a concentration in a range of from about 0.05% to about 0.25%, about 0.06% to about 0.2%, or about 0.08% to about 0.16% (w/v) in a buffer or other suitable aqueous composition according to the disclosure.

Suitable zwitterionic surfactants that can be used according to the methods described herein include betaine zwitterionic surfactants such alkyl betaines, alkylamidobetaines, amidazoliniumbetaines, sulfobetaines (INCI Sultaines), as well as a phosphobetaines.

Examples of suitable zwitterionic surfactants include alkyl betaines, such as those of the formula:

R^{1′}—N⁺(CH₃)₂—CH₂COO⁻;

R^{1′}—CO—NH(CH₂)₃—N⁺(CH₃)₂—CH₂COO⁻;

R^{1′}—N⁺(CH₃)₂—CH₂CH(OH)CH₂SO₃⁻; or

R^{1′}—CO—NH—(CH₂)₃—N⁺(CH₃)₂—CH₂CH(OH)CH₂SO₃⁻.

Examples of suitable betaines and sulfobetaines are the following (designated in accordance with INCI): almondamidopropyl betaine, apricotamidopropyl betaine, avocadamidopropyl betaine, babassuamidopropyl betaine, behenamidopropyl betaine, behenyl betaine, canolamidopropyl betaine, capryl/capramidopropyl betaine, camitine, cetyl betaine, cocamidoethyl betaine, cocamidopropyl betaine, cocamidopropyl hydroxysultaine, coco betaine, coco hydroxysultaine, coco/oleamidopropyl betaine, coco sultaine, decyl betaine, dihydroxyethyl oleyl glycinate, dihydroxyethyl soy glycinate, dihydroxyethyl stearyl glycinate, dihydroxyethyl tallow glycinate, dimethicone propyl of PG-betaine, drucamidopropyl hydroxysultaine, hydrogenated tallow betaine, isostearamidopropyl betaine, lauramidopropyl betaine, lauryl betaine, lauryl hydroxysultaine, lauryl sultaine, milk amidopropyl betaine, milkamidopropyl betaine, myristamidopropyl betaine, myristyl betaine, oleamidopropyl betaine, oleamidopropyl hydroxysultaine, oleyl betaine, olivamidopropyl betaine, palmamidopropyl betaine, palmitamidopropyl betaine, palmitoyl camitine, palm kernel amidopropyl betaine, polytetrafluoroethylene acetoxypropyl betaine, ricinoleamidopropyl betaine, sesamidopropyl betaine, soyamidopropyl betaine, stearamidopropyl betaine, stearyl betaine, tallowamidopropyl betaine, tallowamidopropyl hydroxysultaine, tallow betaine, tallow dihydroxyethyl betaine, undecylenamidopropyl betaine and wheat germ amidopropyl betaine.

Suitable betaine zwitterionic surfactants may be N-(alkyl C₁₀₋₁₆)—N,N-dimethylglycine betaine, N-(alkyl C₁₂₋₁₄)—N,N-dimethylglycine betaine, N,N-dimethyl-N-dodecylglycine betaine, lauryl dimethyl betaine (also known as lauryl betaine), myristyl sulfobetaine, or n-hexadecyl-N,N-dimethyl-3-ammonio-1-propanesulfonate. Lauryl betaine is commercially available as EMPIGEN BB® (Huntsman Corporation) and has a CMC of 1.6-2.1 mM (20-25° C.). Myristyl sulfobetaine (also known as n-tetradecyl-N,N-dimethyl-3-ammonio-1-propanesulfonate, DMMA) is available under the trade name ZWITTERGENT® 3-14 (Merck KGaA, Darmstadt, Germany), and has a CMC 100-400 uM, n-hexadecyl-N,N-dimethyl-3-ammonio-1-propanesulfonate (also known as 3-N,N-dimethylpalmitylammonio)propane sulfonate, DMPA) is available under the tradename ZWITTERGENT® 3-16, and has a CMC 10-60 uM. For example, coconut dimethyl betaine is commercially available from Seppic under the trade name of AMONYL 265®; and lauryl betaine is commercially available from Sigma-Aldrich under the trade name EMPIGEN BB®1. A further example betaine is lauryl-imino-dipropionate commercially available from Rhodia under the trade name MIRATAINE H2C-HA®.

The zwitterionic surfactant can be present in a range of from about 0.06% to about 0.2%, or about 0.08% to about 0.16% in a buffer or other suitable aqueous composition according to the disclosure.

Examples of suitable anionic surfactants include sarcosinate surfactants in acidic form or in neutral form. For example, suitable anionic surfactants may be sarcosinate surfactants in neutral form. Sarcosinate surfactants may be alkanoyl sarcosinate surfactants.

Examples of suitable anionic surfactants include surfactants of the general formula R^{1′}[CO—X(CH₂)ⱼ]_{g}—(CH₂)_{f}—[CH(OH)CH₂]—Y⁻, wherein R^{1′} is a saturated or unsaturated C₅₋₂₄ alkyl, such as a C₈₋₁₈ alkyl, a saturated C₁₀₋₁₆ alkyl or a saturated C₁₂₋₁₄ alkyl; X is NH, NR^{4′}, wherein R^{4′} is C₁₋₄ alkyl, O or S; j is an integer from 1 to 10, such as from 2 to 5 and 3; g is 0 or 1; f is an integer from 0 to 4, such as 0, 1, 2, 3, or 4; h is 0 or 1; and Y is COO, SO₃, OPO(OR^{5′})O or P(O)(OR^{5′})O, wherein R^{5′} is H or C₁₋₄ alkyl, and wherein the anionic surfactant may be in acidic form, or sodium, or potassium salt forms thereof.

Suitable anionic surfactants may comprise the structure CH₃(CH₂)ₐCH₂(CH₂CH═CH)_{b}CH₂(CH₂)_{c}CH₂(C═O)N(CH₃)CH₂CO₂X, wherein a=1-8; b=0-2, and c=0-6, and X═H, Na, K.

Examples of alkanoyl sarcosinates, may include those of the formulae:

R^{1′}—CO—N(CH₃)—CH₂—COO⁻; and

R^{1′}—CO—N(CH₃)—CH₂—SO₃⁻, and, for example, sodium or potassium salts thereof,
wherein R^{1′} may be saturated or unsaturated C₅₋₂₄ alkyl, C₇₋₁₉ alkyl, or C₁₁₋₁₇ alkyl.

Examples of suitable alkanoyl sarcosinates, and acidic or salt forms thereof include N-lauroyl sarcosine, sodium lauroylsarcosinate, sodium palmitoyl sarcosinate, sodium stearoyl sarcosinate, N-methyl-N-(1-oxotetradecyl)-glycine sodium salt, sodium caproyl sarcosinate, sodium capryloyl sarcosinate, N-methyl-N-(1-oxo-9-octadecen-1-yl)-glycine, sodium salt, sodium oleoyl sarcosinate, and sodium linoleoyl sarcosinate.

The anionic surfactant can be present in a range of from about 0.06% to about 0.2%, or about 0.08% to about 0.16% in a buffer or other suitable aqueous composition according to the disclosure.

The compositions can be used in flow cytometry and, as such, can comprise additional components, including, but not limited to, one or more of any suitable carrier, stabilizer, buffer, salt, chelating agent (e.g., EDTA) or preservative. The compositions can also comprise one or more additional surfactants in addition to the zwitterionic surfactants and/or anionic surfactants described herein. Non-limiting examples of the one or more additional surfactants includes polysorbates such as TWEEN® 20 (polyoxyethylene sorbitan monolaurate) and TWEEN® 80 (polyoxyethylene sorbitan monooleate). The carrier can be an aqueous solution, such as water, saline, alcohol, or a physiologically compatible buffer, such as Hank's solution, Ringers solution, or physiological saline buffer. The carrier may include formulation agents, such as suspending agents, stabilizing agents and/or dispersing agents. The compositions can also include a buffer or pH adjusting agent, and typically the buffer is a salt prepared from an organic acid or base. Representative buffering agents include salts of organic acid salts, such as citric acid, ascorbic acid, gluconic acid, carbonic acid, tartaric acid, succinic acid, acetic acid, or phthalic acid; Tris tromethamine hydrochloride, or phosphate buffer.

The composition can comprise a protein stabilizer selected from the group consisting of a bovine serum albumin (BSA or “Fraction V”), a casein, and a gelatin. The protein stabilizer can be BSA, a commercially available bovine serum albumin protein derived from cows. The protein stabilizer can be present in from about 0.1-5 mg/mL, about 0.5-3 mg/mL, or about 2 mg/mL in a buffer or other suitable aqueous composition according to the disclosure.

The stabilizer can be a gelatin, a protein, commonly derived from collagen taken from animal body parts. It is brittle when dry and gummy when moist. It may also be referred to as hydrolyzed collagen, collagen hydrolysate, gelatine hydrolysate, hydrolyzed gelatine, and collagen peptides after it has undergone hydrolysis. Several types of gelatin are commercially available including gelatin-type A, gelatin-type B, Prionex® highly purified gelatin Type A, and gelatin-cold water fish.

The composition can also include any appropriate preservative. The preservative can be an antioxidant, biocide, or antimicrobial agent. The preservative can be an inorganic salt. The preservative can be sodium azide. The preservative may be present in a concentration range of about 0.01 to about 1%, about 0.05% to about 0.5%, or about 0.1%.

### Polymer Dye

In another embodiment, the composition can be used with a polymer dye. The polymer dye may be a fluorescent polymer dye or a fluorescent polymer tandem dye. Polymeric dyes are particularly useful for analysis of chemical and biological target analytes. They are highly responsive optical reporters and efficient light absorbers, by virtue of the multiple chromophores they comprise. The polymer dye conjugate can comprise any fluorescent polymer dye or fluorescent polymer tandem dye previously disclosed.

For example, the polymer dye or tandem polymer dye can be any dye disclosed in Published PCT Appl. No. WO 2017/180998; U.S. Application No. 2021/0047476; U.S. Application No. 2020/0190253; U.S. Application No. 2020/0147615; U.S. Application No. 2021/0108083; U.S. Application No. 2018/0224460; U.S. Pat. Nos. 11,034,840; 10,228,375; 10,545,137B2; 10,533,092; 7,214,489; 8,354,239; 8,575,303, each of which are incorporated by reference as if fully set forth herein in their entirety. The polymer dye conjugate can have the structure of any water-soluble fluorescent polymer dye disclosed in Published US Appl. No. 2020/0190253 A1, which is incorporated by reference as if fully set forth herein in its entirety. The polymer dye conjugate can have the structure of any water-soluble fluorescent polymer dye disclosed in Published US Appl. No. 2019/0144601, which is incorporated by reference as if fully set forth herein in its entirety.

The polymer dye or polymer dye conjugate can be any commercially available polymer dye or polymer dye conjugated to a binding partner. The polymer dye or polymer dye conjugate may comprise a polymer dye excitable by a violet laser. The polymer dye or polymer dye conjugate may comprise a polymer dye excitable by a violet laser, for example, at 405 nm. The polymer dye or polymer dye conjugate may comprise a violet laser (405 nm)-excitable polymer dye.

In some embodiments, the polymer dye or polymer dye conjugate may comprise a SuperNova™ dye (Beckman Coulter, Inc.). SuperNova™ polymers are a new generation of polymer dyes useful for flow cytometry application. The polymer dye or polymer dye conjugate may comprise SuperNova™ v428, SuperNova™ v605 or SuperNova™ v786 (Beckman Coulter, Inc.). SuperNova™ v428 has unique photo-physical properties leading to extremely bright conjugates when conjugated to antibodies or other binding partners. For example, SuperNova™ v428 (SN v428) (Beckman Coulter, Inc.) is a polymer dye optimally excited by the violet laser (e.g., 405 nm) with an excitation maximum of 414 nm, an emission peak of 428 nm, and can be detected using a 450/50 bandpass filter or equivalent.

SuperNova™ v428 is one of the brightest dyes excitable by the violet laser, so it is particularly suited for assessing dimly expressed markers. SuperNova™ conjugated antibodies may include anti-CD19 antibody-SuperNova™ v428, anti-CD22 antibody-SuperNova v428, anti-CD25 antibody-SuperNova™ v428, and anti-CD38 antibody-SuperNova™ v428 antibody-polymeric dye conjugates.

SuperNova™ v605 and SuperNova™ v786 (Beckman Coulter, Inc.) are tandem polymer dyes, derived from the core SuperNova™ v428 polymer dye. Both share same absorbance characteristics, with maximum excitation at 414 nm. With SuperNova™ v605 and SuperNova™ v786 having emission peak's at 605 nm and 786 nm, respectively, they are optimally detected using the 610/20 and 780/60 nm bandpass filters of the flow cytometer. SuperNova™ v605 and SuperNova™ v786 may be conjugated, for example, with anti-CD19 antibody, anti-CD22 antibody, anti-CD25 antibody, and anti-CD38 antibody.

The polymer dye or polymer dye conjugate may comprise a polymer dye excitable by an ultra-violet (“UV”) laser. The polymer dye or polymer dye conjugate may comprise a polymer dye excitable by a UV laser at a wavelength of 320 nm to 380 nm, 340 nm to 360 nm, 345 nm to 356 nm, or less than or equal to 380 nm but greater than or equal to 320 nm. The polymer dye or polymer dye conjugate may comprise a UV-excitable polymer dye. The UV-excitable polymer dye or polymer dye conjugate may emit light typically at a wavelength of 380 nm to 430 nm, 406 nm to 415 nm, or less than or equal to 430 nm but greater than or equal to 380 nm.

The polymer dye or polymer dye conjugate can comprise a Brilliant Violet™ dye (BioLegend®/Sirigen Group Ltd.), such as Brilliant Violet 421™ (excitation max. 405 nm, emission max. 421 nm, 450/50 filter), Brilliant Violet 510™ (excitation max 405 nm, emission max 510 nm, 510/50 filter), Brilliant Violet 570™ (excitation max 405 nm, emission max 570 nm, 585/42 filter), Brilliant Violet 605™ (excitation max 405 nm, emission max 603 nm, 610/20 filter), Brilliant Violet 650™ (excitation max 405 nm, emission max 645 nm, 660/20 filter), Brilliant Violet 711™ (excitation max 405 nm, emission max 711 nm, 710/50 filter), Brilliant Violet 750™ (excitation max 405 nm, emission max 750 nm, 780/60 filter), Brilliant Violet 785™ (excitation max 405 nm, emission max 785 nm, 780/60 filter). The polymer dye or polymer dye conjugate may comprise a Spark Violet™ 538 (BioLegend, Inc.)(excitation max 405 nm, emission max 538 nm).

The polymer dye or polymer dye conjugate may comprise a Super Bright dye (Invitrogen, ThermoFisher Scientific). Super Bright dyes may be excited by the violet laser (405 nm). The Super Bright dye may be Super Bright 436 (excitation max 414 nm, emission max 436 nm, 450/50 bandpass filter), Super Bright 600 (emission max 600 nm, 610/20 bandpass filter), Super Bright 645 (emission max 645 nm, 660/20 bandpass filter), or Super Bright 702 (emission max 702 nm, 710/50 bandpass filter).

The polymer dye or polymer dye conjugate may comprise a BD Horizon Brilliant™ Violet polymer dye (Becton, Dickinson and Co., BD Life Sciences). The polymer dye may be a BD Horizon Brilliant™ BV421 (450/40 or 431/28 filter), BV480 (525/40 filter), BV510 (525/40 filter), BV605 (610/20 filter), BV650 (660/20 filter), BV711 (710/50 filter), BV786 (786/60 filter).

The polymer dye may be prepared synthetically by polymerization of monomers, which leads to formation of a highly conjugated fluorescent backbone. Capping may be carried out on the polymer by activation using appropriate functionalities, which results in a polymer capable of being conjugated to a binding partner. Alternatively, the polymer may be activated for conjugation by attaching appropriate functionalities off the polymer backbone. The activated polymers may be conjugated to a binding partner. Any appropriate binding partner may be employed, for example, an antibody, followed by purification, for example, by using standard procedures. Functional groups can be selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to a substrate or binding partner.

The polymer dye conjugate can comprise fluorescent polymers having monomer subunits including, but not limited to, dihydrophenanthrene (DHP), fluorene, and combinations thereof. In some embodiments, the polymer dye conjugate can comprise a polymer dye having the structure of Formula III:

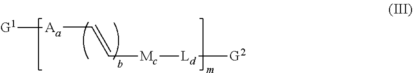

Each A is independently selected from the group consisting of an aromatic co-monomer and a heteroaromatic co-monomer. Each A can be substituted with a functional group that will be conjugated with a binding partner.

Each optional M is independently selected from the group consisting of an aromatic co-monomer, a heteroaromatic co-monomer, a bandgap-modifying monomer, optionally substituted ethylene, and ethynylene, and is evenly or randomly distributed along the polymer main chain. Each M may be independently selected from the group consisting of

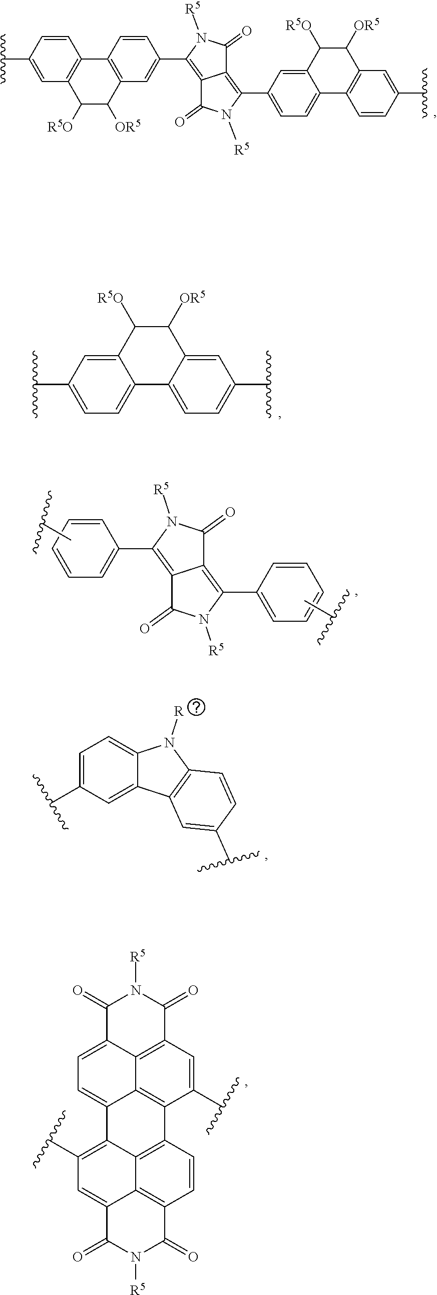

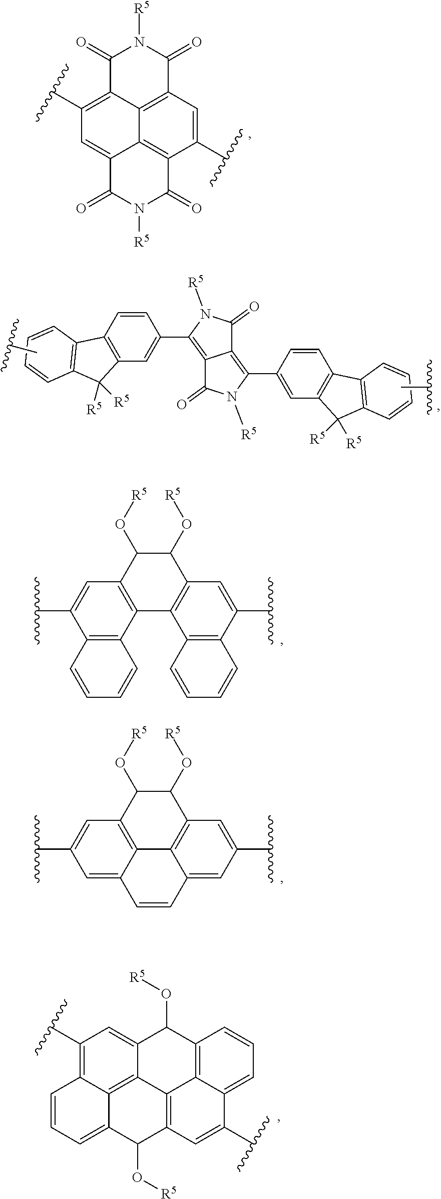

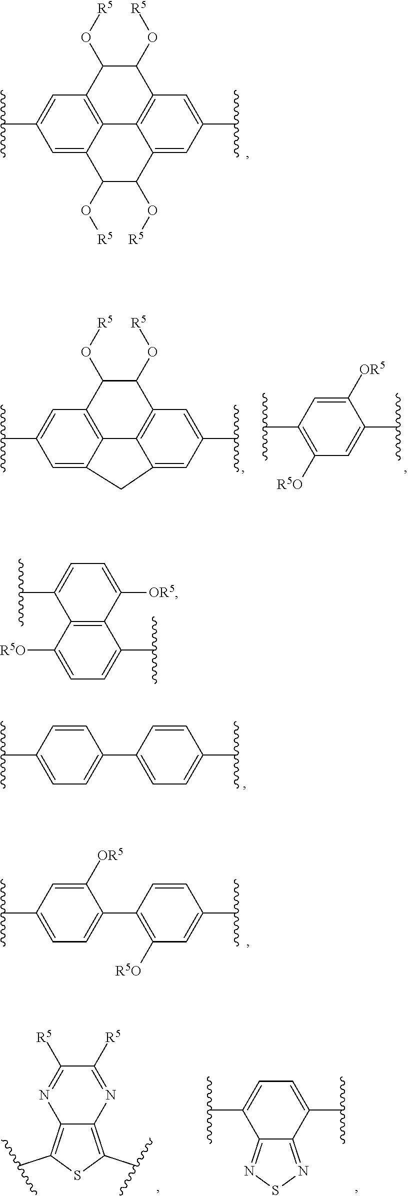

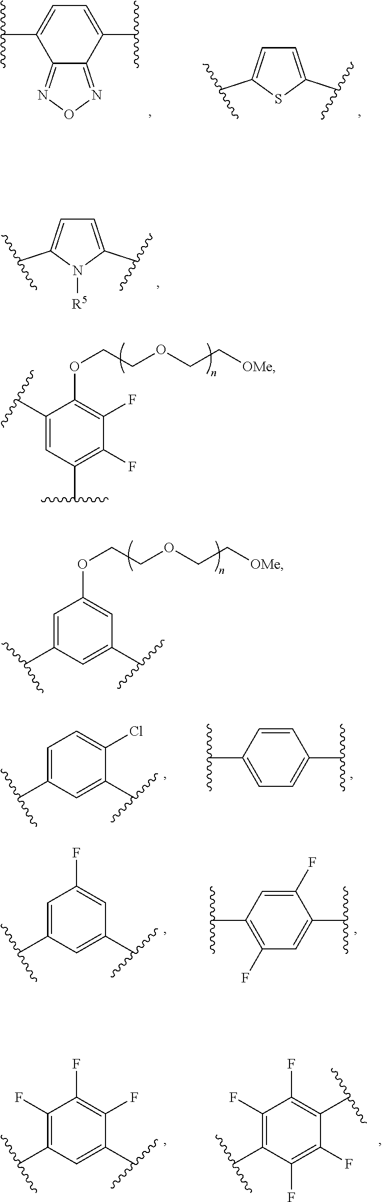

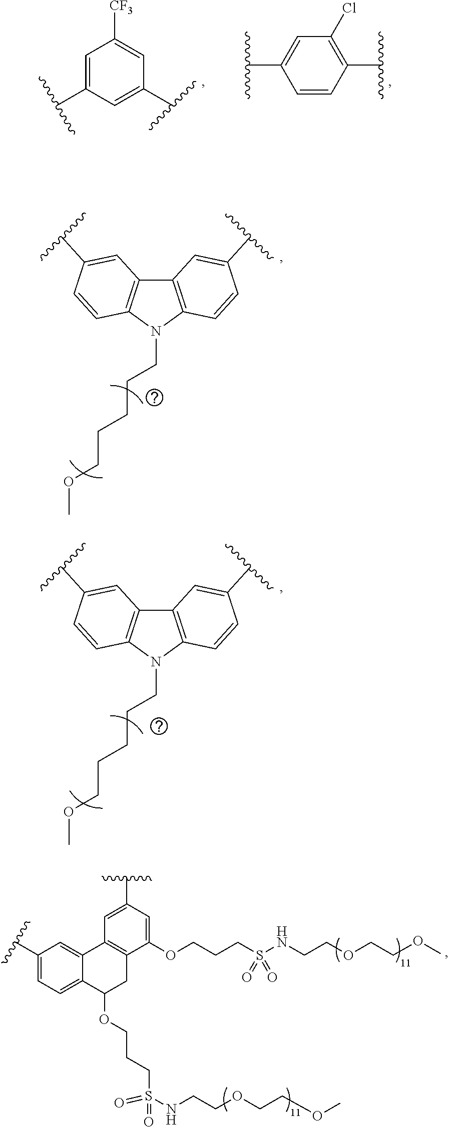

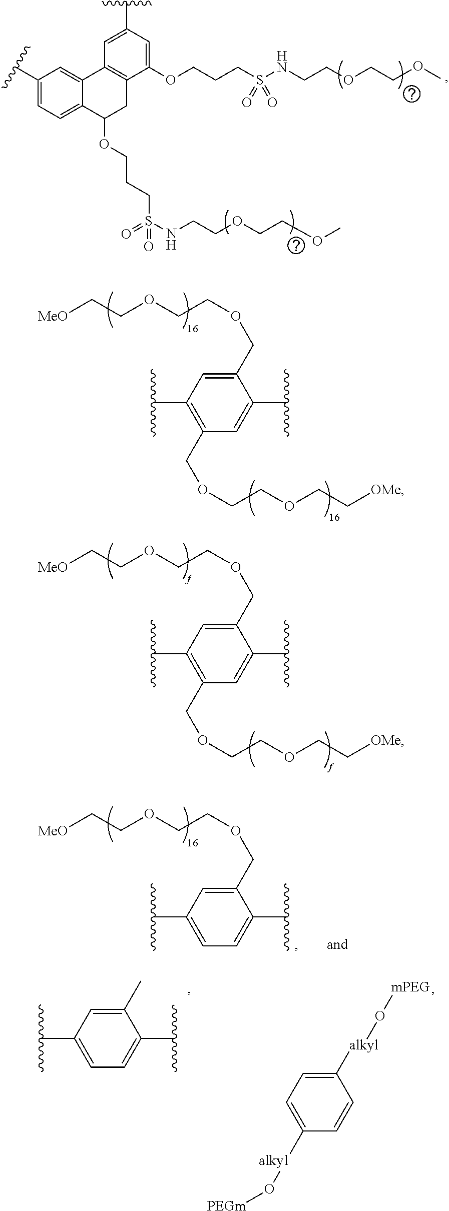

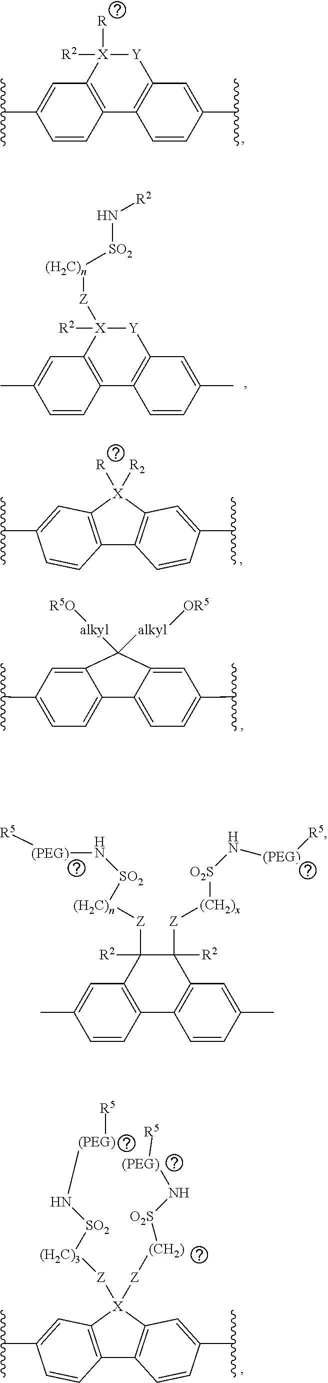

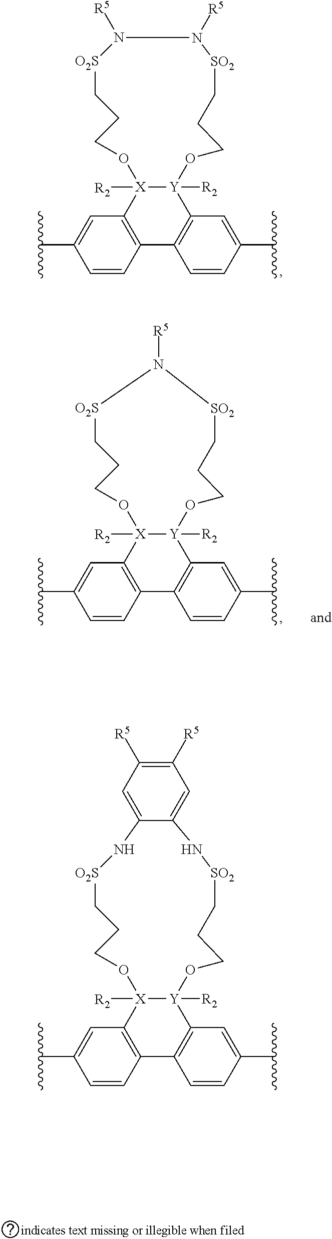

wherein, each M can be substituted, and terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, amide, sulfonamide, ether, thioether, thiocarbamate, hydroxyl, iodoacetyl, hydrazido, hydrazino, ketone, phosphine epoxide, urea, thiourea, thioester, imine, disulfides, and protected groups thereof for conjugation to another substrate, acceptor dye, molecule or binding agent, and
where, each R⁵ is independently selected from the group consisting of halogen, hydroxyl, C₁-C₁₂alkyl, C₂-C₁₂ alkene, C₂-C₁₂ alkyne, C₃-C₁₂ cycloalkyl, C₁-C₁₂haloalkyl, C₁-C₁₂ alkoxy, C₂-C₁₈ (hetero)aryl group, C₂-C₁₈ (hetero)aryloxy, C₂-C₁₈ (hetero)arylamino, carboxylic acid, carboxylate ether, (CH₂)_{x′}(OCH₂—CH₂)_{y′}OCH₃, and (CH₂)_{x′}(OCH₂—CH₂)_{y′}OCF₃ where each x′ is independently an integer from 0-20 and each y′ is independently an integer from 0-50; wherein
each R¹ is independently an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety:

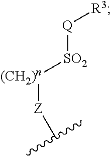

each R² is independently H, alkyl, alkenyl, alkynyl, cycloalkyl, haloalkyl, alkoxy, (hetero)aryloxy, aryl. (hetero)arylamino, a PEG group, an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety

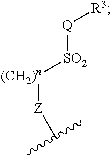

each R³ is independently selected from the group consisting of H, alkyl, alkene, alkyne, cycloalkyl, haloalkyl, alkoxy, (hetero)aryloxy, aryl, (hetero)arylamino, and a PEG group;
each Z is independently selected from the group consisting of C, O, and N;
each Q is independently selected from the group consisting of a bond, NH, NR⁴, and CH₂; and
each subscript n is independently an integer from 0 to 20.

Linkers are represented in Formula III as L Each optional linker L may be an aryl or heteroaryl group evenly or randomly distributed along the polymer main chain and can be substituted with one or more pendant chains terminated with a functional group selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to a substrate or binding partner.

The polymers complexes of the disclosure also contain terminus represented in Formula III as each G¹ and G². The terminus may be modified or unmodified. The terminus may each independently selected from the group consisting of hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted dihydrophenanthrene (DHP), optionally substituted fluorene, aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof that may be conjugated to a substrate or binding partner.

In the structure of Formula III, a, c, and d define the mol % of each unit which each can be evenly or randomly repeated and where each a is a mol % from 10 to 100%, each c is a mol % from 0 to 90%, and each d is a mol % from 0 to 25%; each b is independently 0 or 1; and each m is an integer from 1 to about 10,000.

In some embodiments, the polymer dye conjugate can have the structure of Formula I:

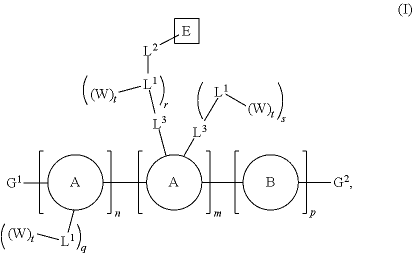

wherein:
each A is independently selected from the group consisting of an aromatic co-monomer and a heteroaromatic co-monomer;
L¹, L², and L³ are linker moieties;
W is a water-solubilizing moiety;
each E is an independently selected chromophore, functional moiety, or binding partner;
each B is independently selected from the group consisting of an aromatic co-monomer, a heteroaromatic co-monomer, a bandgap-modifying monomer, optionally substituted ethylene, and ethynylene;
G¹ and G² are independently selected from an unmodified polymer terminus and a modified polymer terminus;
subscripts n and m are independently integers ranging from 1 to 10,000,
subscript p is an integer ranging from 0 to 10,000, and
the sum of subscripts n, m, and p ranges from 2 to 10,000;
subscript q is 1, 2, 3, or 4;
subscript r is 1, 2, 3, or 4;
subscript s is 0, 1, 2, or 3;
subscript t is 1 or 2
the sum of subscript r and s ranges from 1 to 4; and
A and B are distributed randomly or non-randomly in the conjugated polymer.
L¹ can be a sulfonamide, a sulfonamide, a sultam, a disulfenamide, an amide, a phosphonamide, a phosphonamidate, a phosphinamide or a secondary amine. Or
L¹ can be a sulfonamide, an amide, a phosphonamide, or a secondary amine.
The subscript q can be equal to the sum of subscripts r and s, subscript r can be 1 or 2, if subscript r is 1, then subscript s is 0 or 1, and if subscript r is 2, then subscript s is 0.
Each L³ can be a covalent bond.

The conjugated polymer can have a structure according to Formula II:

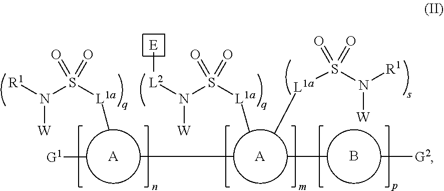

wherein:
L^{1a} is a linker moiety; and
R¹ is selected from the group consisting of H and an amine protecting group.
A variety of linkers L^{1a} and L², as described herein, can be employed for synthesis of polymers according to Formula I and Formula II. For example:
L^{1a} can be selected from the group consisting of a covalent bond, C₁₋₈ alkylene, 2- to 8-membered heteroalkylene (e.g., a divalent alkoxy linker), C₃₋₈ cycloalkylene, C₆₋₁₀ arylene, 5- to 12-membered heteroarylene, 5- to 12-membered heterocyclylene, —NHC(O)L^{a}-, —C(O)NHL^{a}-, —C(O)L^{a}-, and combinations thereof;
L² can be selected from the group consisting of a covalent bond, C₁₋₈ alkylene, 2- to 8-membered heteroalkylene (e.g., a divalent alkoxy linker), C₃₋₈ cycloalkylene, C₆₋₁₀ arylene, 5- to 12-membered heteroarylene, 5- to 12-membered heterocyclylene, -L^{b}NHC(O)—, -L^{b}C(O)NH—, -L^{b}C(O)—, —C(O)NHL^{b}-, —C(O)L^{b}-, and combinations thereof;
L^{a} and L^{b} can be independently selected from the group consisting of C₁₋₈ alkylene and 2- to 8-membered heteroalkylene; and
R¹ can be selected from the group consisting of H and an amine protecting group.
Polymers according to Formula II are provided wherein:
L^{1a} is selected from the group consisting of a covalent bond, C₁₋₈ alkylene, 2- to 8-membered heteroalkylene, —NHC(O)L^{a}-, —C(O)NHL^{a}-, and —C(O)L^{a}-,
L² is selected from the group consisting of a covalent bond, C₁₋₈ alkylene; 2- to 8-membered heteroalkylene, -L^{b}NHC(O)—, -L^{b}C(O)NH—, -L^{b}C(O)—, —C(O)NHL^{b}-, and —C(O)L^{b}-;
L^{a} and L^{b} are independently selected from the group consisting of C₁₋₈ alkylene and 2- to 8-membered heteroalkylene; and
R¹ is selected from the group consisting of H and an amine protecting group.
W can comprise one or more ethylene glycol monomers. Or W can comprise poly(ethylene glycol).
L³ can be a trivalent arylalkyl moiety having: a first point of attachment to a first L¹ moiety (or a first L^{1a} moiety); a second point of attachment to a second L¹ moiety (or a second L^{1a} moiety); and a third point of attachment to an A monomer.

For example, the disclosure provides conjugated polymers having two or more chromophores attached as shown in Formula VI:

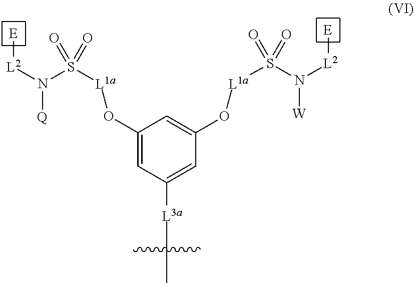

Wherein
L^{1a} is as previously defined;
L² is as previously defined;
W is as previously defined;
L^{3a} is selected from the group consisting of a covalent bond. C₁₋₈ alkylene, 2- to 8-membered heteroalkylene, —NHC(O)L^{a}-, —C(O)NHL^{a}-, and —C(O)L^{a}-;
L^{a} is selected from the group consisting of C₁₋₈ alkylene and 2- to 8-membered heteroalkylene; and the wavy line is the point of the attachment to the a monomer.
Each A monomer in polymers having a structure of Formula I, II or III can be the same monomer. Each A monomer in polymers having a structure of Formula I, II or III can be a different monomer. A can be a fluorescent monomer. A can be a 9,10-phenanthrenedione-based monomer (e.g., a dihydrophenanthrene (DHP)-based monomer), a fluorene-based monomer, or a fluorenooxepine-based monomer.

Monomers A in polymers having a structure of Formula I, II or III can be DHP-based monomers such as:

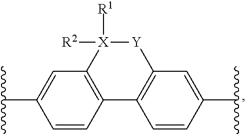

wherein:
each X is independently C or Si;
each Y is independently CR¹R² or SiR¹R²;
each R¹ is independently an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety:

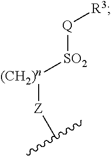

each R² is independently H, alkyl, alkenyl, alkynyl, cycloalkyl, haloalkyl, alkoxy, (hetero)aryloxy, aryl, (hetero)arylamino, a PEG group, an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety

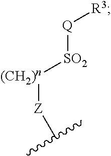

each R³ is independently selected from the group consisting of H, alkyl, alkene, alkyne, cycloalkyl, haloalkyl, alkoxy, (hetero)aryloxy, aryl, (hetero)arylamino, and a PEG group; each Z is independently selected from the group consisting of C, O, and N; each Q is independently selected from the group consisting of a bond, NH, NR⁴, and CH₂; and
each subscript n is independently an integer from 0 to 20.

R¹ can have the structure shown below, wherein Q is NH:

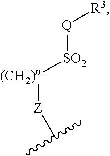

each R³ is independently selected from the group consisting of H, alkyl, alkene, alkyne, cycloalkyl, haloalkyl, alkoxy, (hetero)aryloxy, aryl, (hetero)arylamino, and a PEG group; and each Z is independently selected from the group consisting of C, O, and N.

The DHP-based monomer can have the structure.

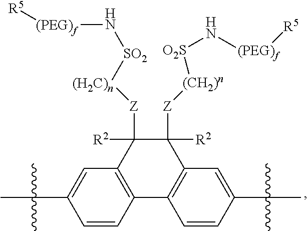

wherein:
each subscript f is independently an integer from 0 to 50;
each subscript n is independently an integer from 0 to 20;
each R² is independently H, alkyl, alkenyl, alkynyl, cycloalkyl, haloalkyl, alkoxy, (hetero)aryloxy, aryl, (hetero)arylamino, a PEG group, an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety

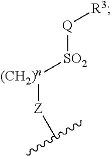

each R⁵ is independently H, C₁-C₂₀ alkyl, C₂-C₂₀ alkenyl, C₂-C₂₀ alkynyl, C₃-C₂₀ cycoalkyl, C₁-C₂₀ haloalkyl, C₁-C₂₀ alkoxy, C₂-C₂₆ aryloxy, C₂-C₂₆ heteroaryloxy, C₂-C₂₆ arylamino, or C₂-C₂₆ heteroarylamino; and
each Z is independently selected from the group consisting of C, O, and N.

The DHP monomer can have the structure:

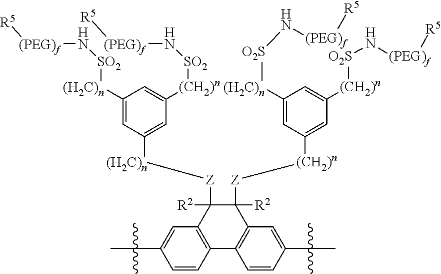

wherein:
each subscript f is independently an integer from 0 to 50;
each subscript n is independently an integer from 0 to 20;
each R² is independently H, alkyl, alkenyl, alkynyl, cycloalkyl, haloalkyl, alkoxy, (hetero)aryloxy, aryl, (hetero)arylamino, a PEG group, an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety

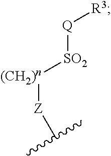

each R⁵ is independently H, C₁-C₂₀ alkyl, C₂-C₂₀ alkenyl. C₂-C₂₀ alkynyl, C₃-C₂₀ cycloalkyl, C₁-C₂₀ haloalkyl, C₁-C₂₀ alkoxy, C₂-C₂₆ aryloxy, C₂-C₂₆ heteroaryloxy, C₂-C₂₆ arylamino, or C₂-C₂₆ heteroarylamino; and
each Z is independently selected from the group consisting of C, O, and N.

Monomers A in polymers having a structure of Formulas I, II or Ill can be fluorene-based monomers such as:

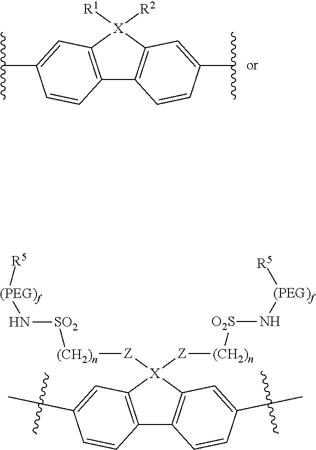

wherein X, Z, R¹, R², R⁵, subscript n, subscript f are as defined herein.

R¹ groups and R² groups such as ammonium alkyl salts, ammonium alkyloxy salts, ammonium oligoether salts, sulfonate alkyl salts, sulfonate alkoxy salts, sulfonate oligoether salts, sulfonamido oligoethers, or moieties having the structure:

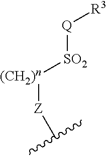

can impart solubility in water/buffer. In some embodiments, for example, the polymer is soluble at levels in excess of 10 mg/mL, in excess of 15 mg/mL, in excess of 20 mg/mL, in excess of 25 mg/mL, in excess of 30 mg/mL, in excess of 35 mg/mL, in excess of 40 mg/mL, in excess of 45 mg/mL, in excess of 50 mg/mL, in excess of 60 mg/mL, in excess of 70 mg/mL, in excess of 80 mg/mL, in excess of 90 mg/mL or in excess of 100 mg/mL.

Monomers A also include bridged monomers. For example, bridged monomers of the present invention include:

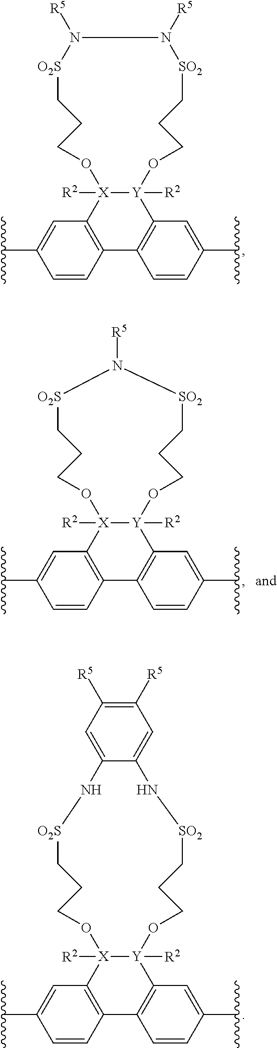

wherein, X, Y, R², and R⁵ are as previously defined.

Monomers A in polymers having a structure of Formula I, II or III can be oxepine-based monomers (e.g., fluorenooxepine-based monomers), such as:

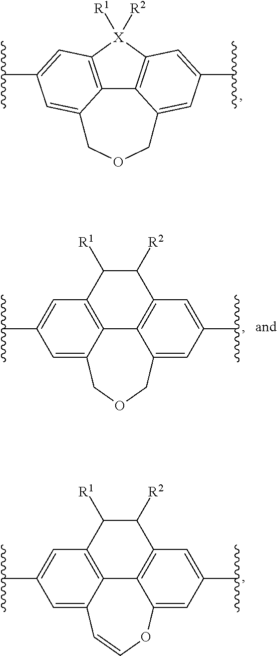

wherein X, R¹, and R² are as defined herein.

### Tandem Polymer Dyes

The polymer can have acceptor dyes attached to the backbone that will provide for monitoring the emission of the acceptor dyes attached to the backbone through energy transfer. Acceptor dyes useful in the tandem polymer dyes include, for example, FITC, CY3B, Cy55, Alexa 488, Texas red, Cy5, Cy7, Alexa 750, and 800CW. For example, acceptor dyes can be attached to the polymer through a linker L:

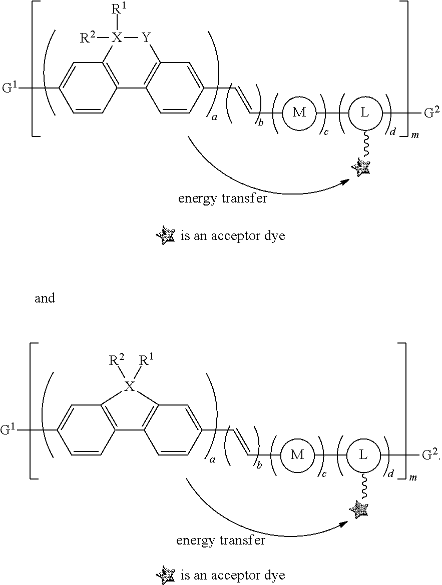

As described in US Published Application No. 2020/0190253, which is incorporated herein by reference in its entirety, acceptor dyes can also be attached directly to monomer A as group E in the structures of Figure I or II above. SuperNova tandem dyes SuperNova v605 and SuperNova v786 (Beckman Coulter, Inc.) are tandem polymer dyes, derived from the core SuperNova v428. Both SuperNova v605 and SuperNova v786 share the same absorbance characteristics, with maximum excitation at 414 nm. With emission peak respectively at 605 nm and 786 nm, they are optimally detected using the 610/20 and 780/60 nm bandpass filters of the flow cytometer.

### Conjugate Dyes

The polymer dyes may be conjugated to different specificities of binding partners, e.g., target-analyte specific antibodies, in order to synthesize a binding partner-dye conjugate such as CD19-SN v428, CD20-SN v605, etc.

The polymer dye and polymer dye conjugates may be formulated with an aqueous buffer. Any appropriate aqueous buffer may be employed, for example, an isotonic aqueous buffer such as a PBS buffer. The aqueous buffer may include additives. For example, the aqueous buffer may include BSA, sodium azide, a non-ionic surfactant, e.g. PF-68, and a zwitterionic surfactant, e.g., Empigen BB®, or anionic surfactant, e.g., NLS, as described herein. BSA helps in stabilizing the conjugate, sodium azide prevents from any microbial contamination, and the surfactant, such as Empigen BB®, significantly reduces or eliminates non-specific binding on the monocytes & granulocytes. The BSA may be present in a range of from 0-3 mg/mL, 0.5-2.5 mg/mL or about 2 mg/mL. The sodium azide may be present in a range of from 0-0.05%, 0.05-0.03%, or about 0.01% (w/v).

### Binding Partner

As used herein, “binding partner” refers to any molecule or complex of molecules capable of specifically binding to a target analyte. The binding partner may be, for example, a protein (e.g., an antibody or an antigen-binding antibody fragment), a small organic molecule, a carbohydrate (e.g., a polysaccharide), an oligonucleotide, a polynucleotide, a lipid, an affinity ligand, an aptamer, or the like. In some embodiments, the binding partner is an antibody or fragment thereof. Specific binding in the context of the present invention refers to a binding reaction which is determinative of the presence of a target analyte in the presence of a heterogeneous population. Thus, under certain assay conditions, the specified binding partners bind preferentially to a particular protein or isoform of the particular protein and do not bind in a significant amount to other proteins or other isoforms present in the sample.

In some cases, the antibody includes intravenous immunoglobulin (IVIG) and/or antibodies from (e.g., enriched from, purified from, e.g., affinity purified from) IVIG. IVIG is a blood product that contains IgG (immunoglobulin G) pooled from the plasma (e.g., in some cases without any other proteins) from many (e.g., sometimes over 1,000 to 60,000) normal and healthy blood donors. IVIG is commercially available. Aspects of IVIG are described, for example, in US. Pat. Appl. Pub. Nos. 2010/0150942; 2004/0101909; 2013/0177574; 2013/0108619; and 2013/0011388, which are incorporated herein by reference.

When the binding partners are antibodies, they may be monoclonal or polyclonal antibodies. The term “antibody” as used herein refers to immunoglobulin molecules and immunologically active portions of immunoglobulin (Ig) molecules, for example, which specifically bind to an antigen in a target analyte. Such antibodies include, but are not limited to, polyclonal, monoclonal, mono-specific polyclonal antibodies, antibody mimics, chimeric, single chain, Fab, Fab′ and F(ab)₂ fragments, Fv, and an Fab expression library. In some cases, the antibody is a monoclonal antibody of a defined sub-class (e.g., IgG1, IgG2, IgG3, or IgG4, IgA, IgD, IgE, IgG2a, IgG2b, IgG3, and IgM). If combinations of antibodies are used, the antibodies can be from the same subclass or from different subclasses. For example, the antibodies can be IgG1 antibodies. In some embodiments, the monoclonal antibody is humanized. Antibody fragments may include molecules such as Fab, scFv, F(ab′)2, and Fab′ molecules. Antibody derivatives include antibodies or fragments thereof having additions or substitutions, such as chimeric antibodies. Antibodies can be derived from human or animal sources, from hybridomas, through recombinant methods, or in any other way known to the art.

Binding partners other than antibodies or target analyte specific antibody fragments or derivatives can also be used in the present system and methods. For example, binding partners may be nucleic acids or nucleic-acid analogs, such as oligonucleotides or PNA probes. In one embodiment, aptamers can be used as specific binding partners. Aptamers are single-stranded DNA or RNA (ssDNA or ssRNA) molecules that can bind to pre-selected targets including proteins and peptides with high affinity and specificity. Other binding partners that can bind to target analyte to form pairs of receptor-ligand, enzyme-substrate, enzyme-inhibitor, and enzyme-cofactor pairs can also be used. Specific examples of such binding partner pairs include carbohydrate and lectin, biotin and avidin or streptavidin, folic acid and folate binding protein, vitamin B12 and intrinsic factor, Protein A and immunoglobulin, and Protein G and immunoglobulin. Also included are binding partners that form a covalent bond with the target analytes.

### Conjugation

A polymer dye conjugate can comprise any known polymer dye conjugated to a binding partner using techniques known to those of skill in the art. In some embodiments, a polymer dye can be conjugated to a binding partner to form a polymer dye conjugate using the method of direct modification of core polymers described in US Published Application No. 2020/0190253, which is incorporated herein by reference in its entirety.

In some instances, a polymer dye can be conjugated to a binding partner to form a polymer dye conjugate using the method described in US Published Application No. 2019/0144601, which is incorporated herein by reference in its entirety. The method can be depicted as follows:

SuperNova v428 (SN v428 (Beckman Coulter) is a bright polymer dye that can be activated with amine for tandem dyes, followed by maleimide activation for tandem conjugates. The rigidity of the polymer dye structure may help reduce rotational energy leading to brighter emissions. SuperNova v428 is one of the brightest dyes excitable by the violet laser, so it is particularly suited for assessing dimly expressed markers. SuperNova conjugated antibodies may include anti-CD19 antibody-SuperNova v428, anti-CD22 antibody-SuperNova v428, anti-CD25 antibody-SuperNova v428, and anti-CD38 antibody-SuperNova v428 antibody-polymeric dye conjugates.

### Target Analyte

The disclosure also relates to a method for detecting a target analyte in a sample, wherein the target analyte comprises a target antigen and can be a substance, e.g., molecule, whose abundance/concentration is determined by some analytical procedure. The present invention is designed to detect the presence, and in some cases the quantity of specific target analytes. The term target analyte refers to a target molecule containing a target antigen to be detected in a biological sample, for example, peptides, proteins, polynucleotides, organic molecules, sugars and other carbohydrates, lipids, and small molecules. It is an important aspect of the disclosure that the target analytes are comprised in a liquid sample and are accessible, or made accessible at some point, to bind target analyte-specific binding partners of the instant invention. Target analytes may be found in a biological sample, such as a blood sample, a cell line development sample, a tissue culture sample, and the like.

The target analyte may be, for example, nucleic acids (DNA, RNA, mRNA, tRNA, or rRNA), peptides, polypeptides, proteins, lipids, ions, monosaccharides, oligosaccharides polysaccharides, lipoproteins, glycoproteins, glycolipids, or fragments thereof. The target analyte can be a protein and can be, for example, a structural microfilament, microtubule, and intermediate filament proteins, organelle-specific markers, proteasomes, transmembrane proteins, surface receptors, nuclear pore proteins, protein/peptide translocases, protein folding chaperones, signaling scaffolds, ion channels and the like. The protein can be an activatable protein or a protein differentially expressed or activated in diseased or aberrant cells, including but not limited to transcription factors, DNA and/or RNA-binding and modifying proteins, nuclear import and export receptors, regulators of apoptosis or survival and the like.

Target analytes can be present and accessible on the surface of cells. Illustrative examples of useful analytes include, but are not limited to, the following: 1) specific cell surface macromolecules and antigens (including hormones, protein complexes, and molecules recognized by cell receptors) and 2) cellular proteins, DNA or RNA in permeabilized cells including abnormal DNA or RNA sequences or abnormal amounts of certain messenger RNA. Detection of these analytes may be particularly useful in situations where they are contained in and/or are identifiers of rare cells such as are found in the early stages of a variety of cancers.

In some examples, the target analyte may be a CD2, CD3, CD4, CD8, CD10, CD11c, CD14, CD15, CD16, CD19, CD20, CD22, CD25, CD27, CD38, CD45, CD45RA, CD56, CD62L, CD64, CD95, CD103, HLA-DR, IFN-γ, TNF-α, or ZAP-70, or other target analyte of interest.

### Biological Sample

Non-limiting examples of the biological sample include blood, serum, plasma, urine, semen, milk, sputum, mucus, a buccal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, samples of in vitro cell culture constituents (including but not limited to conditioned medium resulting from the growth of cells in cell culture medium, putatively virally infected cells, recombinant cells, and cell components).

The sample in the methods of the disclosure can be, for example, blood. The blood sample can be whole blood. The whole blood can be obtained from the subject using standard clinical procedures. The sample can be a subset of one or more cells of whole blood (e.g., erythrocyte, leukocyte, lymphocyte (e.g., T cells, B cells or NK cells), phagocyte, monocyte, macrophage, granulocyte, basophil, neutrophil, eosinophil, platelet, or any cell with one or more detectable markers). The sample can be from a cell culture. The sample may comprise a target analyte naturally or may be prepared through synthetic means, in whole or in part.

### Subject

The subject can be a human (e.g., a patient suffering from, or suspected of suffering from, a disease), a commercially significant food chain mammal, including, for example, a cow, steer, pig, goat, sheep, bird, fish, or horse. Samples can also be obtained from household pets or companion animals, including, for example, a dog, cat, rabbit, bird, or ferret. The subject can be a laboratory animal used as an animal model of disease or for drug screening, for example, a monkey, mouse, a rat, a rabbit, or guinea pig. The subject can be an exotic animal, such as a zoo animal or a wild animal, such as an elephant, antelope, zebra, bison, giraffe, lion, tiger, panther, orangutan, gorilla, whale, dolphin, shark, or reptile.

### Reaction Vessel

A reaction vessel disclosed herein can be any container where reactions between the binding partners or polymer dye conjugates thereof and the target analytes can occur. For example, a reaction vessel can be a tube, a plate, a well of a microtiter plate, a chamber, and a slide. In a preferred embodiment, a reaction vessel has a lid or cap such that the binding reaction can occur in a closed environment.

### Substrate

A reaction vessel comprises one or more substrates. The substrate can be any suitable surface, including but not limited to, plastic, nitrocellulose, cellulose acetate, quartz, and glass. Non-limiting examples of plastic may include polystyrene, polypropylene, cyclo-olefin, and polycarbonate. In some embodiments, the substrate is a membrane. The substrate can be the inside surface of the body of a reaction vessel, e.g., a plastic tube or well of a microtiter plate. The substrate can also be a bead. In some embodiments, at least one of the substrates receiving the labeled binding partners (e.g., a membrane) is bonded to an inside surface of the body of the reaction vessel. In some embodiments, the membrane substrate is a sheet or roll, which makes it easier to deposit the solutions and easier to dry. In some embodiments, the membrane can be cut to separate individual dried reactant spots. In some embodiments, the cut membrane is simply dropped into the reaction vessel. In some preferred embodiments, the cut membranes are bonded to the surface of the reaction vessel, so that the spots do not escape the vessel when liquid is pipetting into or out of the reaction vessel.

### Liquid Sample

The reaction vessel is configured to receive a liquid sample. Liquid samples used in the invention typically comprise target analytes obtained as or dispersed in a predominantly aqueous medium.

The sample can be, for example, a biological sample, such as a blood, bone marrow, spleen cells, lymph cells, bone marrow aspirates (or any cells obtained from bone marrow), urine (lavage), serum, plasma, saliva, cerebral spinal fluid, lymph fluid, urine, amniotic fluid, interstitial fluid, feces, mucus, milk, semen, buccal swab, nasopharangial swab, a vaginal swab, a rectal swab, an aspirate, a needle biopsy, a section of tissue obtained for example by surgery or autopsy, or tissue (e.g., tumor samples, disaggregated tissue, disaggregated solid tumor) sample. The sample can be a blood sample. The blood sample can be a whole blood sample. The whole blood can be obtained from the subject using standard clinical procedures. The sample can be a subset of one or more cells of whole blood (e.g., erythrocyte, leukocyte, lymphocyte (e.g., T cells, B cells or NK cells), phagocyte, monocyte, macrophage, granulocyte, basophil, neutrophil, eosinophil, platelet, or any cell with one or more detectable markers). The sample can be from a cell culture, in vitro cell culture constituents (including but not limited to conditioned medium resulting from the growth of cells in cell culture medium, putatively virally infected cells, recombinant cells, and cell components).

Samples can be any source of biological material, and may include proteins, carbohydrates, and/or polynucleotides that can be obtained from a living organism, directly or indirectly. Samples can include, e.g., cells, tissue, or fluid, and the deposits left by that organism, including viruses, mycoplasma, and fossils. The sample may comprise a target analyte. The target analyte may be naturally occurring in a biological sample, or may be prepared through synthetic means, in whole or in part.

### Labeled Binding Partner

Dyes can be conjugated to binding partners by various linking chemistry between reactive pairs located in the binding partners and the labels. The reactive pairs can include, but not limited to, maleimide/thiol, succimidylester (NHS ester)/amine, azide chemistry, carboxy/EDC (1-Ethyl-3-[3-dimethylaminopropyl]carbodiimide Hydrochloride)/amine, amine/Sulfo-SMCC (Sulfosuccinimidyl 4-[N-maleimidom ethyl]cyclohexane-1-carboxylate)/thiol, and amine/BMPH (N-[^{˜}-Maleimidopropionic acid]hydrazide.TFA)/thiol. Methods for performing the conjugation are well known in the art. Commercial kits for performing the conjugation are also readily available, e.g., from Innova biosciences (Cambridge, UK), Novus Biologicals (Littleton, Colo.), Thermo Fisher Scientific (Waltham, Mass.).

Either a dry or liquid polymer dye conjugate can be used in the methods and compositions. Dried polymer dye conjugate can be prepared using any technique known in the art. The techniques can be as described in US 2019/0242882, which is incorporated herein by reference.

A polymer dye conjugate may be employed in a composition according to the disclosure that may be used directly to stain blood and analyze it in a flow cytometer.

### Assay Systems

Assay systems utilizing a binding partner and a fluorescent label to quantify bound molecules are well known. Examples of such systems include flow cytometers, scanning cytometers, imaging cytometers, fluorescence microscopes, and confocal fluorescent microscopes.

Flow cytometry is used to detect fluorescence. A number of devices suitable for this use are available and known to those skilled in the art. Examples include BCI Navios, Gallios, Aquios, and CytoFLEX™ flow cytometers.

The assay can be an immunoassay. Examples of immunoassays useful in the invention include, but are not limited to, fluoroluminescence assay (FLA), and the like. The assays can also be carried out on protein arrays.

When the binding partners are antibodies, antibody or multiple antibody sandwich assays can also be used. A sandwich assay refers to the use of successive recognition events to build up layers of various binding partners and reporting elements to signal the presence of a particular analyte. Examples of sandwich assays are disclosed in U.S. Pat. No. 4,486,530 and in the references noted therein.

A light source is applied to the sample that can excite the polymer and light emitted from the conjugated polymer complex is detected. In the typical assay, fluorescent polymer dye conjugates for use in the invention are excitable with a light having wavelength between about 395 nm and about 415 nm. The emitted light is typically between about 415 nm and about 475 nm. Alternatively, excitation light can have a wavelength between about 340 nm and about 370 nm and the emitted light is between about 390 nm and about 420 nm.

### Applications

Compositions according to the disclosure may include a single-color, i.e., a single polymer dye conjugate, such as a single SN polymer dye conjugate. For example, biological samples may be stained using SN conjugates to monitor or identify particular cell populations, depending on the antibody conjugated to the polymer dye.

In some embodiments, compositions according to the disclosure may include a single color polymer dye conjugate along with conventional non-polymeric dye conjugates. For example, SN conjugates can be used along with non-polymeric dye conjugates such as CD4-FITC, CD7-PE, CD25-ECD, CD56-PC5.5, etc., in a panel to identify cell subpopulations in human whole blood samples by flow cytometry.

In some embodiments, one or a plurality of the compositions according to the disclosure may be contacted with a biological sample, such as a blood sample. For example, biological samples may be stained with a composition comprising a plurality of SN conjugates to monitor or identify particular cell populations, depending on the antibody conjugated to the polymer dye. In some embodiments, 2 or more, 3 or more, or 4 compositions according to the invention may be contacted with a biological sample. In some embodiments, compositions comprising a plurality of polymer dye conjugate compositions may further comprise non-polymeric dye conjugates such as CD4-FITC, CD7-PE, CD25-ECD, CD56-PC5.5, etc., in a panel to identify cell subpopulations in human whole blood samples by flow cytometry.

### EXAMPLES

The present invention can be better understood by reference to the following examples which are offered by way of illustration. The present invention is not limited to the examples given herein.

### Example 1: Preparation of DHP Polymer Complex

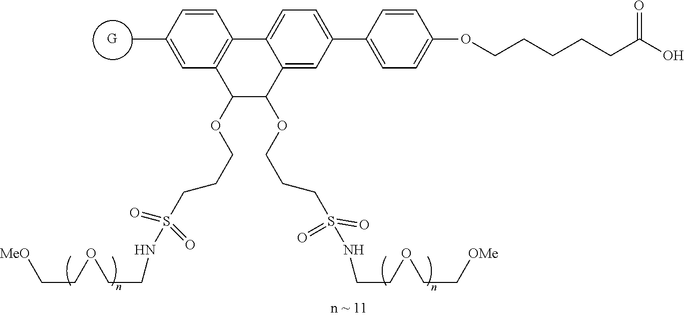

Method 1: In a round bottom flask dibromo DHP monomer and diboronic DHP monomers, as described in WO 2017/180998, (1:1) were taken in (DMF-water) mixture and purged with nitrogen for 10 minutes. Under nitrogen about 20 equivalent of CsF and 10% of Pd(OAc)2 were mixed and heated at 80 deg Celsius. Polymerization was monitored using UV-Vis spectroscopy and SEC chromatography. Later to the reaction mixture, a capping agent (selected from G1) containing appropriate functional group was added and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 2: Alternatively, the polymerization can be done by self-polymerizing a bromo-boronic ester of DHP molecule. In a round bottom flask DHP bromoboronic ester was taken in (DMF-water) mixture and purged with nitrogen for 10 minutes. Under nitrogen about 10 equivalent of CsF and 5% of Pd(OAc)₂ were mixed and heated at 80deg Celsius. Polymerization was monitored using UV-Vis spectroscopy and SEC chromatography. Later to the reaction mixture, a capping agent (selected from G1) containing appropriate functional group was added and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 3. In a round bottom flask both the dibromo dihydrophenanthrene and diboronic dihydrophenanthrene monomers (1:1) were taken and dissolved in THF-water (4:1) mixture containing 10 equivalent of K₂CO₃ and 3% Pd(PPh₃)₄. The reaction mixture was put on a Schlenk line and was degassed with three freeze-pump-thaw cycles and then heated to 80deg C. under nitrogen with vigorous stirring for 18 hours. Later to the reaction mixture, a capping agent (selected from G1) containing appropriate functional group was added via a cannula under excess nitrogen pressure and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 4: Alternatively the polymerization can be done by self-polymerizing a bromo-boronic ester of dihydrophenanthrene molecule. In a round bottom flask dihydrophenanthrene bromoboronic ester was taken and dissolved in THF-water (4:1) mixture containing 10 equivalent of K₂CO₃ and 3% Pd(PPh₃)₄. The reaction mixture was put on a Schlenk line and was degassed with three freeze-pump-thaw cycles and then heated to 80deg C. under nitrogen with vigorous stirring for 18 hours. Later to the reaction mixture, a capping agent (selected from G1) containing appropriate functional group was added via a cannula under excess nitrogen pressure and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

### Example 2: Preparation of Fluorene-DHP Copolymer Complex

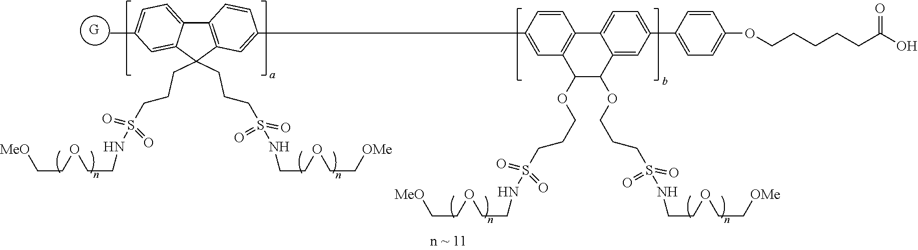

Method 1. In a round bottom flask both the dibromo DHP and diboronic fluorene monomers (1:1) were taken in (DMF-water) mixture and purged with nitrogen for 10 minutes. Under nitrogen about 20 equivalent of CsF and 10% of Pd(OAc)2 were mixed and heated at 80deg Celsius. Polymerization was monitored using UV-Vis spectroscopy and SEC chromatography. Later to the reaction mixture, a capping agent (selected from G1) containing appropriate functional group was added and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 2. In a round bottom flask both the dibromo fluorene and diboronic DHP monomers (1:1) were taken in (DMF-water) mixture and purged with nitrogen for 10 minutes. Under nitrogen about 20 equivalent of CsF and 10% of Pd(OAc)2 were mixed and heated at 80deg celcius. Polymerization was monitored using UV-Vis spectroscopy and SEC chromatography. Later to the reaction mixture, a capping agent (selected from G1) containing appropriate functional group was added and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 3: In a round bottom flask both the dibromo dihydrophenanthrene and diboronic fluorene monomers (1:1) were taken and dissolved in THF-water (4:1) mixture containing 10 equivalent of K₂CO₃ and 3% Pd(PPh₃)₄. The reaction mixture was put on a Schlenk line and was degassed with three freeze-pump-thaw cycles and then heated to 80deg C. under nitrogen with vigorous stirring for 18 hours. Later to the reaction mixture, a capping agent (selected from G1) containing appropriate functional group was added via a cannula under excess nitrogen pressure and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

Method 4: In a round bottom flask dibromo fluorene and diboronic dihydrophenanthrene monomers (1:1) were taken and dissolved in THF-water (4:1) mixture containing 10 equivalent of K₂CO₃ and 3% Pd(PPh3)4. The reaction mixture was put on a Schlenk line and was degassed with three freeze-pump-thaw cycles and then heated to 80deg C. under nitrogen with vigorous stirring for 18 hours. Later to the reaction mixture, a capping agent (selected from G1) containing appropriate functional group was added via a cannula under excess nitrogen pressure and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system equipped with a 100K MWCO membrane. It is washed using 20% ethanol until the absorption of the filtrate diminishes.

### Example 3: Comparison of Fluorescence Emission Spectra

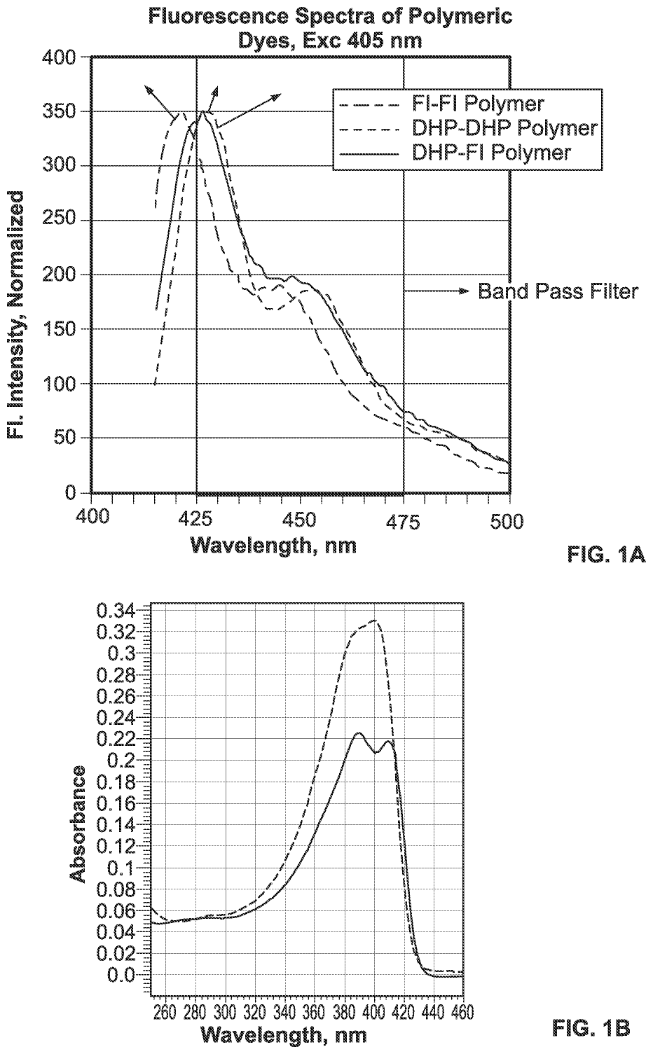

Comparison of fluorescence emission spectra of fluorene (FI-FI), dihydrophenanthrene (DHP-DHP) and fluorene-DHP (DHP-FI) polymers were undertaken. After excitation at 405 nm, DHP containing polymers show a marked difference in their fluorescence maxima which is at 426-428 nm, whereas the fluorene based polymers show a maxima of 421 nm, as shown in FIG. 1A.

### Example 4: Comparison of Absorption Spectra

The absorption spectra of both fluorene (FI-FI) polymer and dihydrophenanthrene (DHP-DHP) polymer were measured. The DHP-DHP polymer (black curve) exhibits lambda max (λmax) at 390 and 410 nm, whereas the FI-FI (grey curve) polymer shows lambda max (λmax) at about 400 nm, as shown in FIG. 1B. Samples were measured under different concentrations.

### Example 5: Polymer Dye Properties

Polymer dyes of the disclosure were found to possess certain physical and chemical characteristics of absorption, fluorescence, brightness, molecular weight, polydispersity, dye to protein ratio when conjugated to an antibody etc. The preferred ranges of these parameters are shown in Table 1A.

TABLE 1A
Polymer Dye Characteristics

| Abs/Em Max | MW (Mₙ) | PD | ε | ϕ | Brightness | F/P |
|---|---|---|---|---|---|---|
| λₘₐₓ395-415 nm | 20K-70K | Between 1.5 | 300K to | 0.4 to 0.75 | 150K to 1900K | 1 to 12 |
| λₑₘ 420-430 nm | | to 2.5 | 2500K | | | |
| λₘₐₓ340-370 nm | 20K-70K | Between 1.5 | 300K to | 0.1 to 0.75 | 30K to 1900K | 1 to 12 |
| λₑₘ 390-420 nm | | to 2.5 | 2500K | | | |

The excitation and emission spectra of tandem polymers was measured. Excitation was carried out at the polymer maxima (405 nm) and the emissions observed from the various acceptor dyes attached to the backbone.

### Example 6: Experiments with Unconjugated Dye

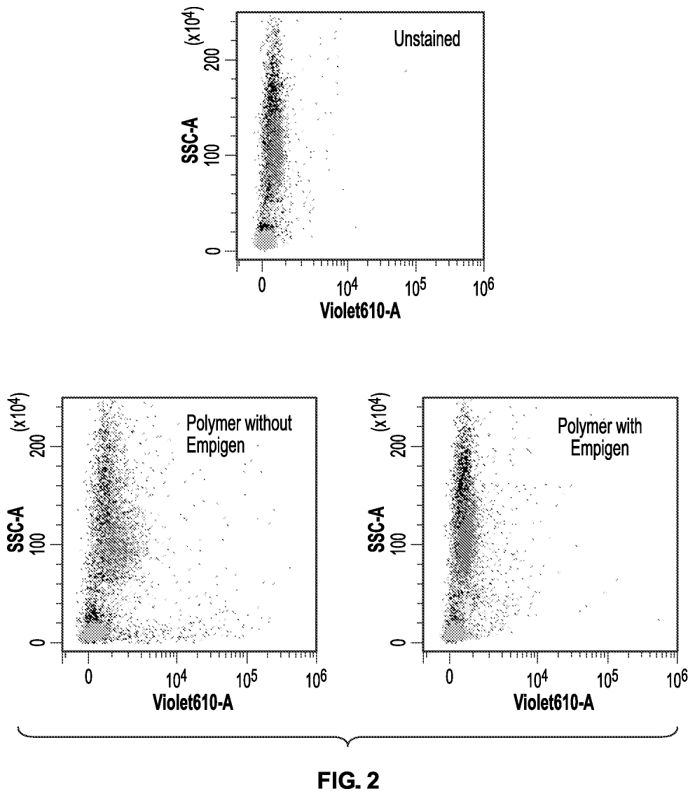

A blood sample was stained with unconjugated polymer dye SN v605 (without antibody), with and without Empigen BB®, and analyzed in a flow cytometer. As shown in FIG. 2, lower right, presence of EMPIGEN BB® showed effective decrease in the non-specific interactions of the fluorescent polymers conjugated to binding partners to white blood cells in blood. In FIG. 2, the fluorescent polymer dye SN v605 without antibody was used to stain a blood sample and analyzed in a flow cytometer. It is evident in FIG. 2, lower left, that the polymer dye without Empigen BB® is binding to the monocytes/granulocytes non-specifically. While not wishing to be bound by any specific theory, it is thought that the polymer is likely adsorbing on the cell surface of monocytes and granulocytes. When EMPIGEN BB® is added, the surface of cells is blocked by EMPIGEN BB® molecules and non-specific binding of polymers to monocytes and granulocytes is substantially reduced.

With the addition of EMPIGEN BB® to the polymer, the dot plots appear like that of an unstained sample of FIG. 2, upper panel, which does not contain any polymer. The spread of the granulocyte population is comparable to unstained tube suggesting that when EMPIGEN BB® is added to the polymer dye the non-specific interaction of the polymer dye with monocytes and granulocytes is reduced drastically.

### Example 7: Experiments with Conjugated Dye SN 605-CD20

EMPIGEN BB® was formulated with the conjugates described herein (e.g., SN605-CD20, SN786-CD103, and SN428 conjugates), bovine serum albumin (BSA; 2 mg/mL), sodium azide (0.1%), and pluronic F-68 (polyethylene oxide-polypropylene oxide-polyethylene oxide nonionic triblock copolymer) to a dose of 0.12% per 10 μl of conjugate.

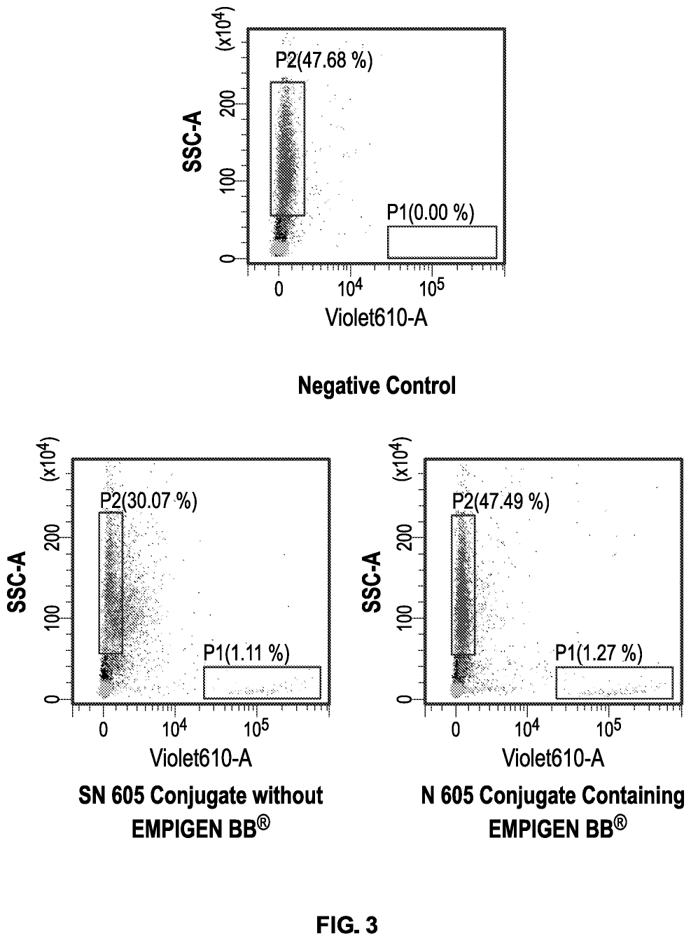

CD20 is a B-lineage cell marker expressed during pre-B lymphocyte development, persists in B-lymphocyte expression, and losses its expression while plasma cell differentiation. CD20 is not expressed on other leukocyte population including monocytes, granulocytes and NK cells. FIG. 3 gives the performance of SN 605-CD20 conjugate without EMPIGEN BB® and in the presence of EMPIGEN BB®.

The percentage of non-specifically bound granulocytes was reduced (see the “P2” gate in the dot plot) with the usage of EMPIGEN BB®). Also, the functional aspect of the conjugate also did not change (see the “P1” gate in the dot plot), since the percentage of the positive population is similar in both the cases.

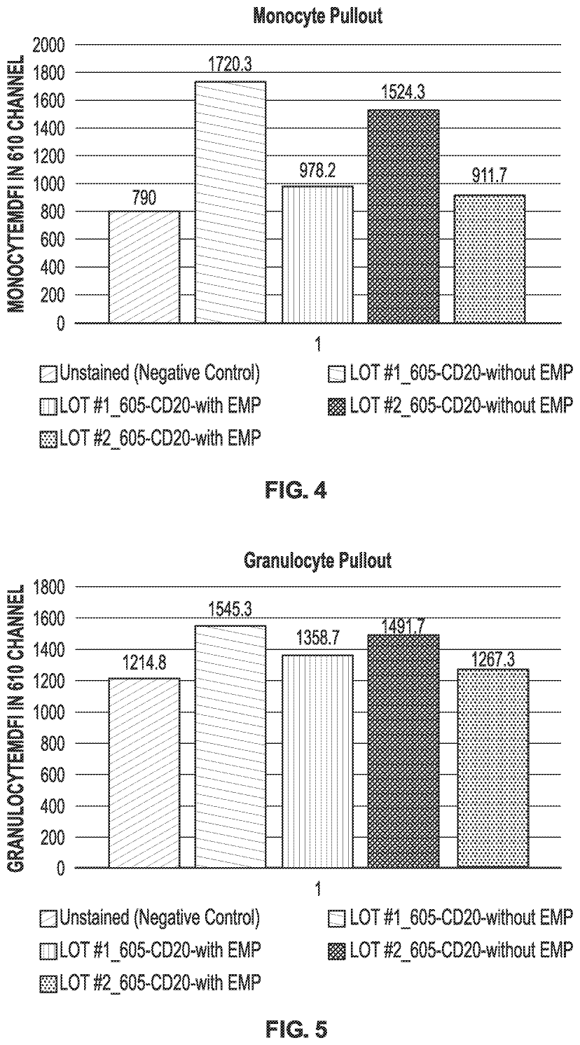

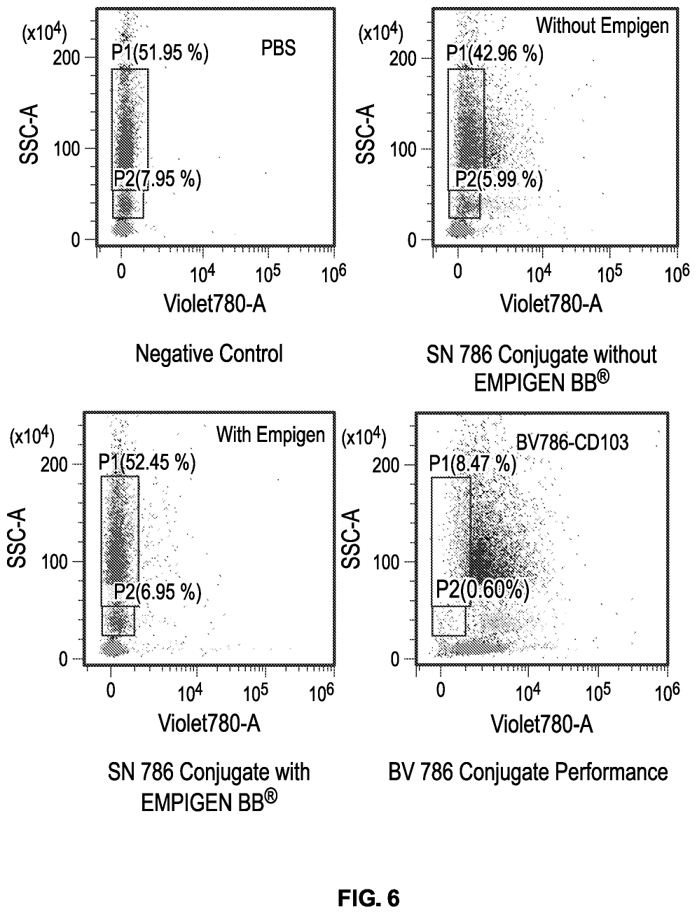

In order to confirm the effect of EMPIGEN BB®, two lots of SN 605-CD20 conjugates were tested in the presence and absence of EMPIGEN BB®. The mean fluorescence intensity (MFI) was compared to the autofluorescence (negative population median fluorescence intensity (MdFI)) of the monocytes from the unstained sample. The results shown in FIG. 4 demonstrate that in the presence of surfactant the non-specific interaction on monocytes were reduced to 75% and 67% for Lot-1 and Lot-2 SN605 CD20 conjugates, respectively. A similar effect (reduction of non-specific binding), was also observed in granulocytes too, but the percent of reduction is not as pronounced (13.7% and 17.7% for Lot-1 and Lot-2 respectively, FIG. 5).

### Example 8: Experiments with Conjugated Dye SN 786-CD103

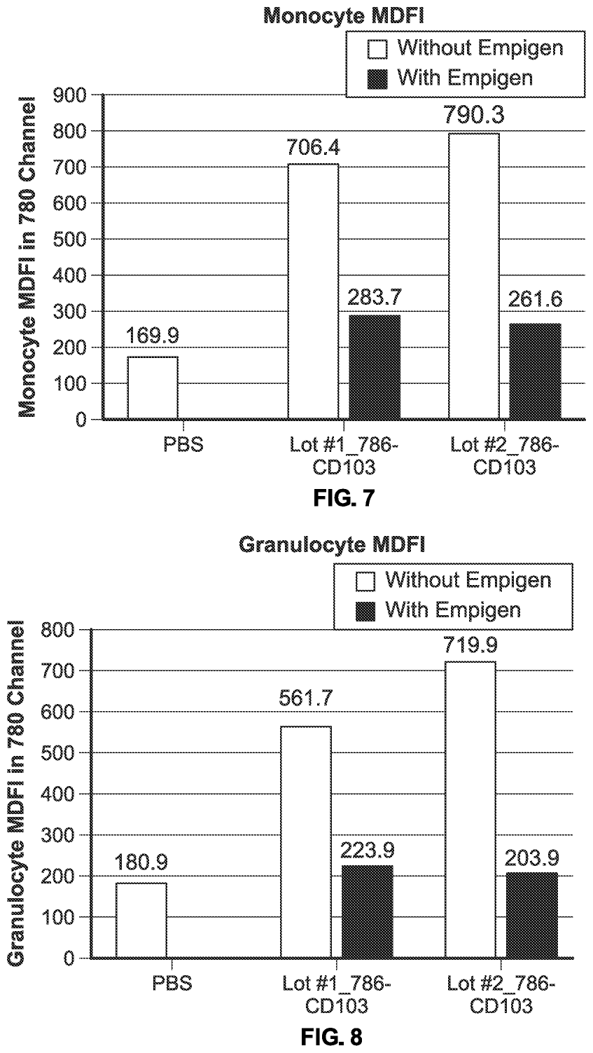

CD103 conjugates are tested on cell line (MOLT16) as they are not usually expressed in normal whole blood. Since there is no expression of CD103 in normal blood, there should be no positive signal. But due to the non-specific interaction, SN786 CD103 conjugates tend to bind to whole blood as well. Addition of EMPIGEN BB® to this formulation helps to contain this non-specific interaction. This is illustrated in FIG. 6.

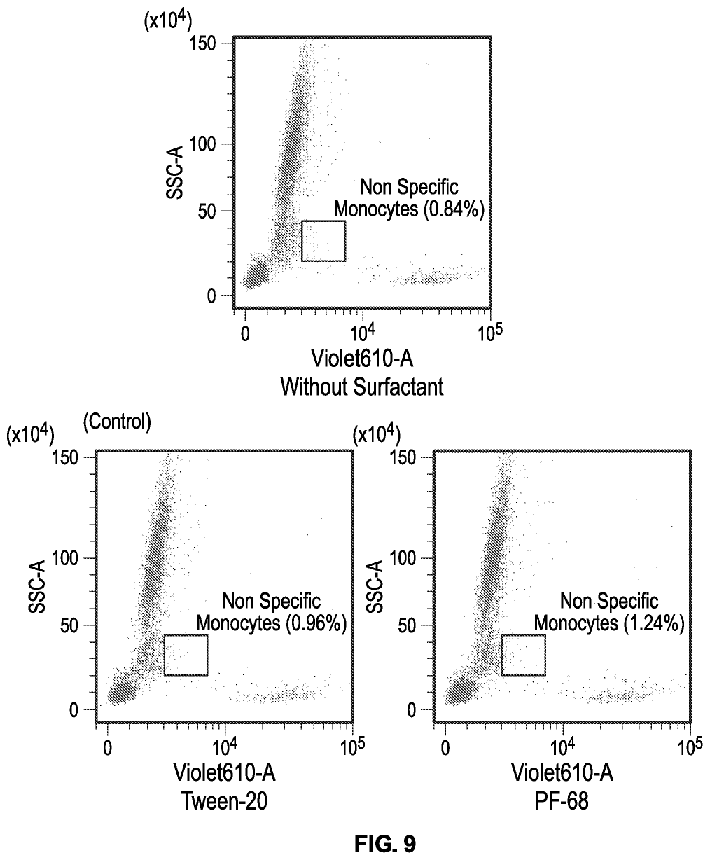

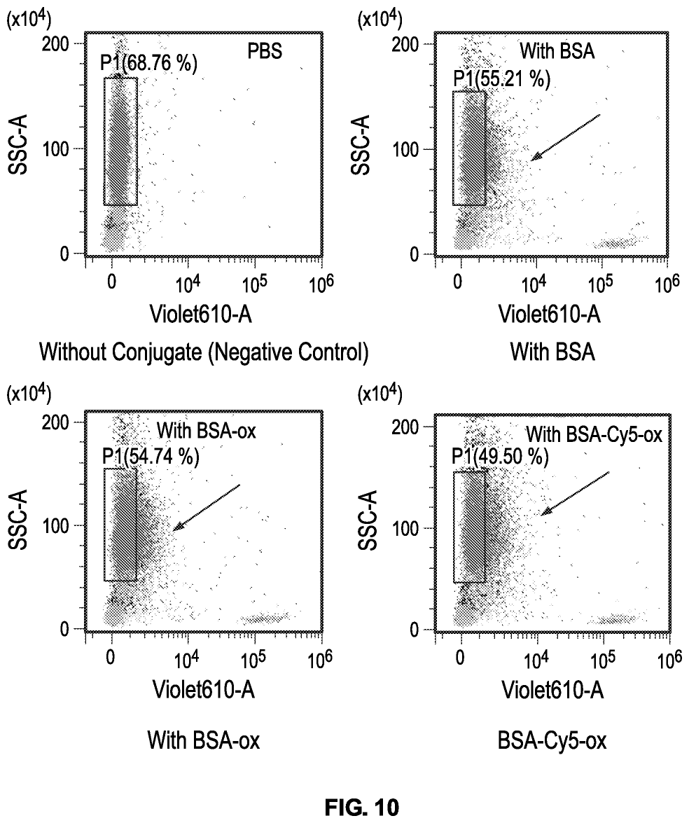

Two lots of SN 786-CD103 conjugates were tested in presence and absence of EMPIGEN BB® and the autofluorescence (negative population MdFI) of the monocyte and granulocyte population is compared against an unstained sample. As shown in FIG. 7, the signal from non-specific binding of conjugates to monocytes reduced 149.3% and 202.1% for Lot-1 and Lot-2 of SUPERNOVA™ conjugates respectively. The same effect (reduction of non-specific binding), was also observed in the granulocyte population as well, with a 150.8% and 253% reduction for Lot-1 and Lot-2 respectively as shown in FIG. 8.

### Example 9: Zwitterionic Surfactant Effect on Monocyte Background Reduction with SN 428 Conjugates

The efficiency of zwitterionic surfactant EMPIGEN BB® on the reduction of non-specific binding with monocytes was evaluated. Other populations were also studied, namely lymphocytes and granulocytes, to evaluate the non-impact of the detergent on MFI and percentage of cells.

The experimental conditions were generally as follows:
CD19-SN 428 (lot D19-094, polymer lot RDS-042919 (82.7 kD), 1 dose (0.5 μg/test).
CD22-SN 428 (lot D19-109, polymer lot WX-20190624 (86.4 kD), 1 dose (0.5 μg/test).
CD25-SN 428 (lot D19-107, polymer lot RDS-062419 (72.8 kD), 1 dose (0.5 μg/test).
CD19, CD22 and CD25-BV 421 from Becton Dickinson at 1× commercial dose.
Three doses of EMPIGEN BB®0.06%, 0.12%, and 0.2% prepared in conjugate final formulation.
Added 10 μL sample with 100 μL whole blood.
Tested on 2 donors lysing with VersaLyse (lysing solution, used to lyse red blood cells, Beckman Coulter, Inc.)+Fix, one wash.
Navios flow cytometer acquisition on FL9.

The required x number of tubes were prepared, wherein “x number of tubes” depends on the performance tested. A calculated volume of conjugated antibody (at required dose) was added to each tube. Whole blood (100 μL) was added in each tube. The tubes were gently vortexed for 15 seconds and incubated for 15 to 20 minutes at room temperature at 18-25° C. and protected from light. VersaLyse and IOTest3 Fixative mixture (2 mL Versalyse Ref. A09777+50 μl IOTest3 fixative 10× Ref. A07800) were added to the tubes. The tubes were immediately vortexed for 1 second and incubated for 10 minutes at room temperature (18-25° C.), protected from light. The tubes were centrifuged for 5 minutes at 300 g at room temperature, the supernatant removed by aspiration, and the cell pellet resuspended using 3 mL of PBS 1×. The tube was again centrifuged for 5 minutes at 300 g at room temperature (18-25° C.), the supernatant removed by aspiration and the cell pellet resuspended using: 0.5 ml PBS 1× or PBS 1× Formaldehyde 0.1% (can be obtained by diluted 1 ml PBS 1×+12.5 μl IOTest3 fixative 10×).

In cytometry, compensation is a mathematical correction of a signal overlap between the channels of the emission spectra of different fluorochromes. Therefore, this correction factor was used to eliminate the bleeding of signals into other unwanted channels. Manual compensation was performed to assess the conjugate performance.

The raw data, normalized data to the condition without EMPIGEN™, and finally the % reduction of monocyte background staining that were obtained on 2 donors with the CD9, CD22 and CD25 SUPERNOVAS v428 conjugates are shown in Table 1B. The data show that in the presence of Empigen BB® the non-specific background monocyte binding was substantially reduced by between 52 and 73% compared to the condition without EMPIGEN® where the monocyte background is maximal.

TABLE 1B
Analysis of background reduction of Empigen on monocytes

| | | Donor 1 | | | Donor 2 | | |
|---|---|---|---|---|---|---|---|
| | | | | % reduction | | | % reduction |
| | | | Normalized | of | | | of |
| | | MFI | to condition | background | MFI | Normalized to | background |
| | | raw | w/o | on | raw | condition w/o | on |
| | | data | Empigen | monocytes | data | Empigen | monocytes |
| CD19 | w/o | 3.380 | 100.0 | 0.0 | 2.290 | 100.0 | 0.0 |
| | Empigen | | | | | | |
| | w/0.06% | 1.600 | 47.3 | 52.7 | 1.250 | 54.6 | 45.4 |
| | Empigen | | | | | | |
| | w/0.12% | 1.090 | 32.2 | 67.8 | 0.950 | 41.5 | 58.5 |
| | Empigen | | | | | | |
| | w/0.20% | 0.890 | 26.3 | 73.7 | 0.770 | 33.6 | 66.4 |
| | Empigen | | | | | | |
| CD22 | w/o | 2.520 | 100.0 | 0.0 | 2.47 | 100.0 | 0.0 |
| | Empigen | | | | | | |
| | w/0.06% | 1.860 | 73.8 | 26.2 | 1.69 | 68.4 | 31.6 |
| | Empigen | | | | | | |
| | w/0.12% | 1.280 | 50.8 | 49.2 | 1.28 | 51.8 | 48.2 |
| | Empigen | | | | | | |
| | w/0.20% | 0.870 | 34.5 | 65.5 | 0.93 | 37.7 | 62.3 |
| | Empigen | | | | | | |
| CD25 | w/o | 2.500 | 100.0 | 0.0 | 2.020 | 100.0 | 0.0 |
| | Empigen | | | | | | |
| | w/0.06% | 1.350 | 54.0 | 46.0 | 1.230 | 60.9 | 39.1 |
| | Empigen | | | | | | |
| | w/0.12% | 0.940 | 37.6 | 62.4 | 0.940 | 46.5 | 53.5 |
| | Empigen | | | | | | |
| | w/0.20% | 0.730 | 29.2 | 70.8 | 0.700 | 34.7 | 65.3 |
| | Empigen | | | | | | |

MFI = Mean Fluorescence Intensity
w/o = without
w/ = with

Data showing the effect of Empigen BB® on granulocyte background reduction is shown in Table 2. Granulocyte background reduction was found to range from 4 to 21% compared to the condition without EMPIGEN® where the granulocyte background is maximal.

TABLE 2
Analysis of background reduction of Empigen on granulocytes

| | | Donor 1 | | | Donor 2 | | |
|---|---|---|---|---|---|---|---|
| | | | | % reduction | | | % reduction |
| | | | Normalized | of | | | of |
| | | MFI | to condition | background | MFI | Normalized to | background |
| | | raw | w/o | on | raw | condition w/o | on |
| | | data | Empigen | monocytes | data | Empigen | monocytes |
| CD19 | w/o | 1.070 | 100.0 | 0.0 | 0.990 | 100.0 | 0.0 |
| | Empigen | | | | | | |
| | w/0.06% | 0.890 | 83.2 | 16.8 | 0.860 | 86.9 | 13.1 |
| | Empigen | | | | | | |
| | w/0.12% | 0.900 | 84.1 | 15.9 | 0.870 | 87.9 | 12.1 |
| | Empigen | | | | | | |
| | w/0.20% | 0.920 | 86.0 | 14.0 | 0.820 | 82.8 | 17.2 |
| | Empigen | | | | | | |
| CD22 | w/o | 1.070 | 100.0 | 0.0 | 0.970 | 100.0 | 0.0 |
| | Empigen | | | | | | |
| | w/0.06% | 1.00 | 93.5 | 6.5 | 0.920 | 94.8 | 5.2 |
| | Empigen | | | | | | |
| | w/0.12% | 0.930 | 86.9 | 13.1 | 0.880 | 90.7 | 9.3 |
| | Empigen | | | | | | |
| | w/0.20% | 0.900 | 84.1 | 15.9 | 0.930 | 95.9 | 4.1 |
| | Empigen | | | | | | |
| CD25 | w/o | 0.930 | 100.0 | 0.0 | 0.880 | 100.0 | 0.0 |
| | Empigen | | | | | | |
| | w/0.06% | 0.890 | 95.7 | 4.3 | 0.840 | 95.5 | 4.5 |
| | Empigen | | | | | | |
| | w/0.12% | 0.750 | 80.6 | 19.4 | 0.810 | 92.0 | 8.0 |
| | Empigen | | | | | | |
| | w/0.20% | 0.730 | 78.5 | 21.5 | 0.730 | 83.0 | 17.0 |
| | Empigen | | | | | | |

Table 3 shows the effect of EMPIGEN® on the positive lymphocyte population: the presence of EMPIGEN® did not induce a significant variation of the positive signal on lymphocytes when compared to the condition without EMPIGEN®.

TABLE 3
Analysis of Empigen effect on positive lymphocytes

| | | Donor 1 | | | Donor 2 | | |
|---|---|---|---|---|---|---|---|
| | | | | % reduction | | | % reduction |
| | | | Normalized | of | | | of |
| | | MFI | to condition | background | MFI | Normalized to | background |
| | | raw | w/o | on | raw | condition w/o | on |
| | | data | Empigen | monocytes | data | Empigen | monocytes |
| CD19 | w/o | 83.25 | 100.0 | 0.0 | 85.06 | 100.0 | 0.0 |
| | Empigen | | | | | | |
| | w/0.06% | 81.91 | 98.4 | −1.6 | 88.21 | 103.7 | 3.7 |
| | Empigen | | | | | | |
| | w/0.12% | 83.28 | 100.0 | 0.0 | 91.85 | 108.0 | 8.0 |
| | Empigen | | | | | | |
| | w/0.20% | 82.36 | 98.9 | −1.1 | 96.72 | 113.7 | 13.7 |
| | Empigen | | | | | | |
| CD22 | w/o | 64.98 | 100.0 | 0.0 | 81.95 | 100.0 | 0.0 |
| | Empigen | | | | | | |
| | w/0.06% | 66.47 | 102.3 | 2.3 | 91.72 | 111.9 | 11.9 |
| | Empigen | | | | | | |
| | w/0.12% | 72.93 | 112.2 | 12.2 | 94.85 | 115.7 | 15.7 |
| | Empigen | | | | | | |
| | w/0.20% | 71.56 | 110.1 | 10.1 | 98.59 | 120.3 | 20.3 |
| | Empigen | | | | | | |
| CD25 | w/o | 6.79 | 100.0 | 0.0 | 5.77 | 100.0 | 0.0 |
| | Empigen | | | | | | |
| | w/0.06% | 6.86 | 101.0 | 1.0 | 5.96 | 103.3 | 3.3 |
| | Empigen | | | | | | |
| | w/0.12% | 6.69 | 98.5 | −1.5 | 6.02 | 104.3 | 4.3 |
| | Empigen | | | | | | |
| | w/0.20% | 6.65 | 97.9 | −2.1 | 6.29 | 109.0 | 9.0 |

Empigen

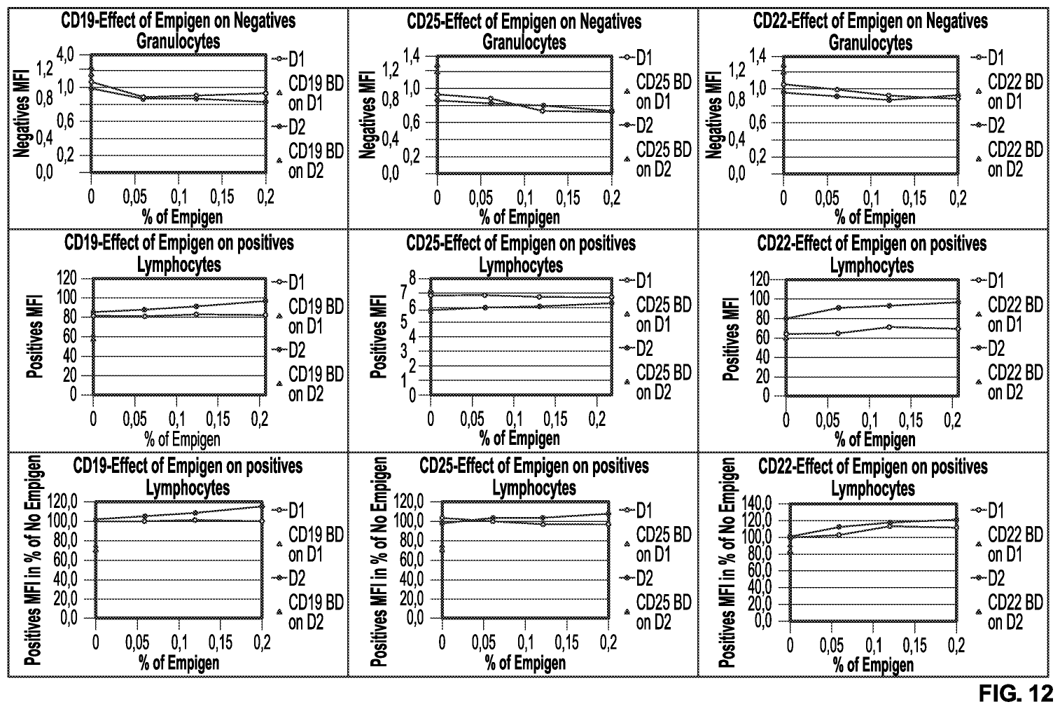

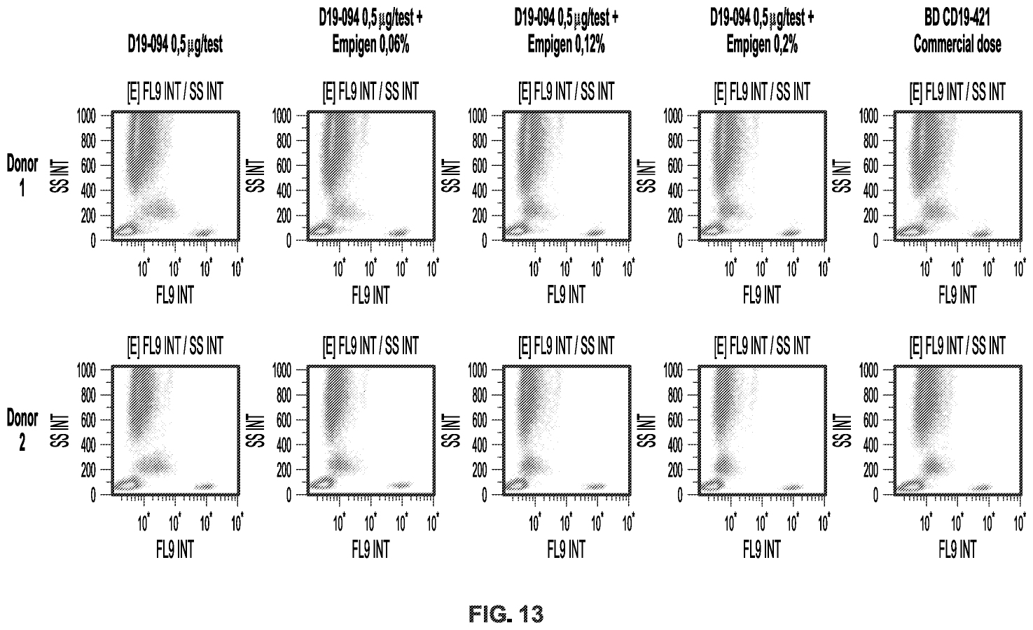

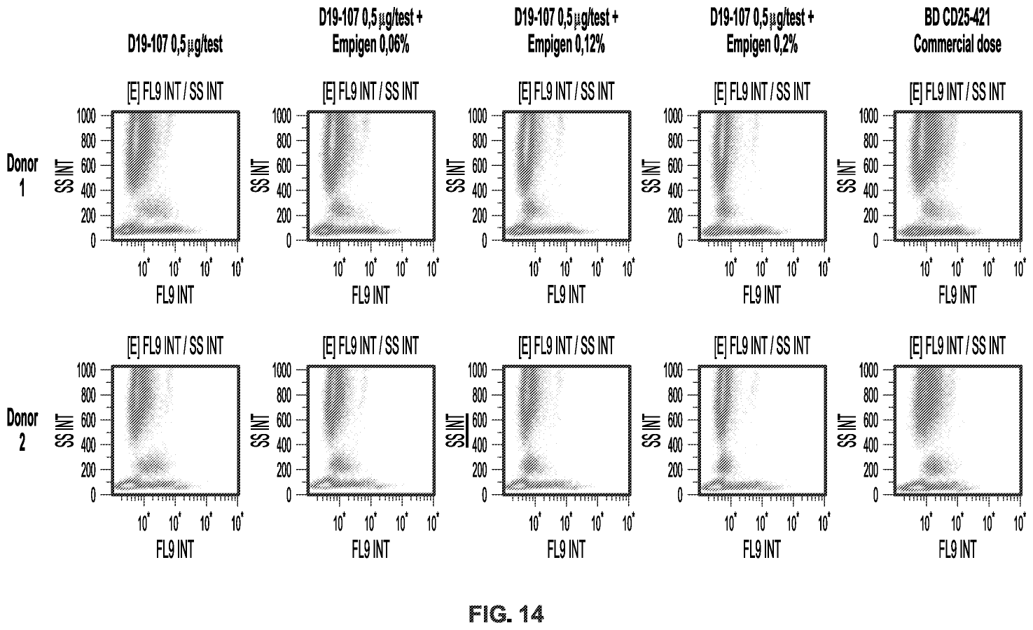

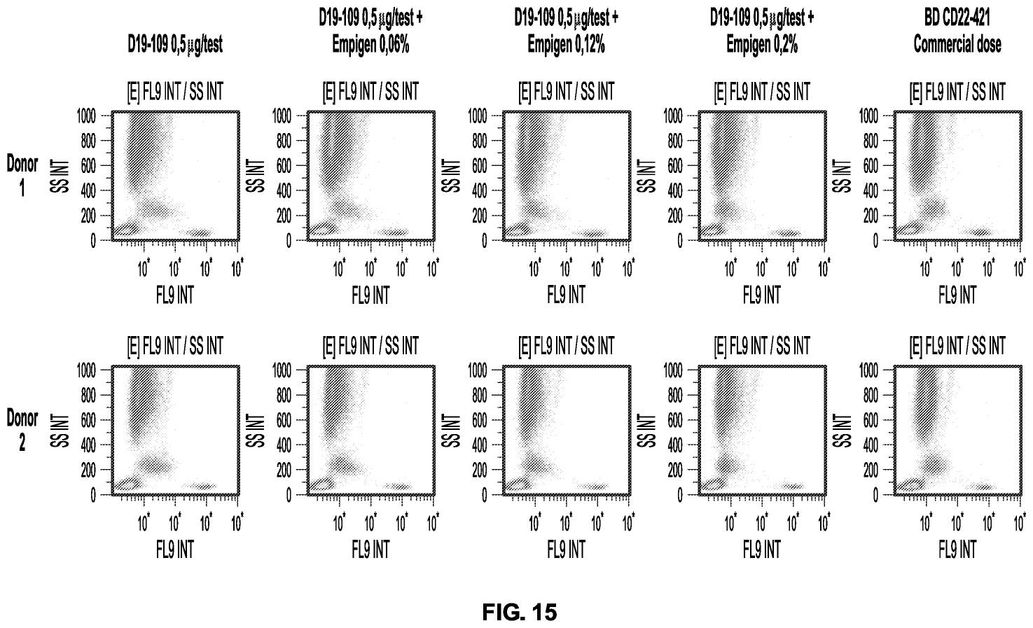

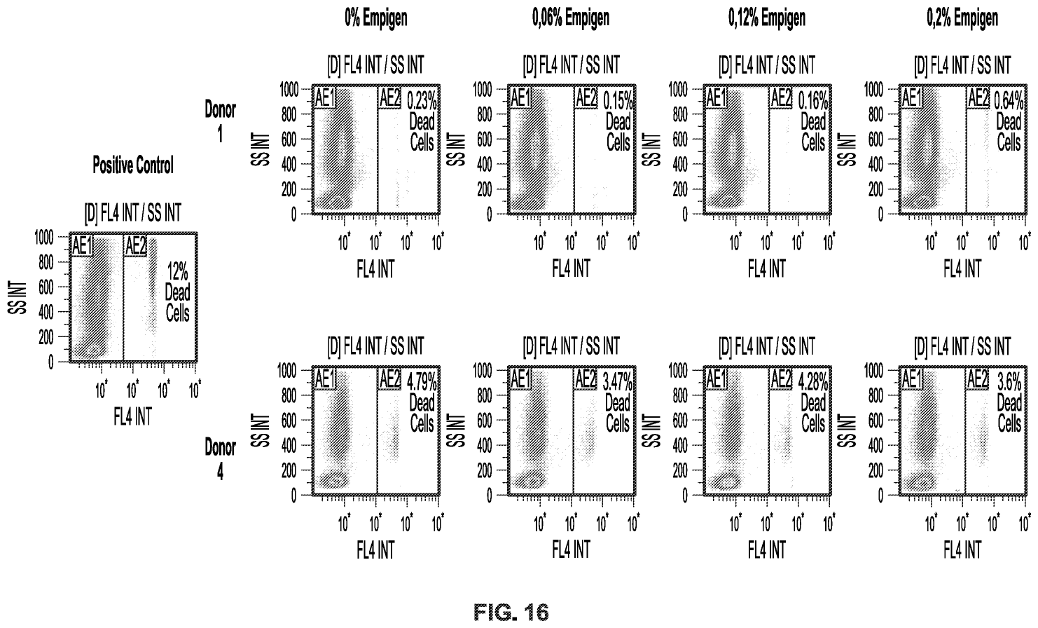

The data in Tables 1-3 are summarized in FIGS. 11-15.

Table 4 describes additional experiments showing percent reduction of background on monocytes and granulocytes.

TABLE 4
Percent reduction of background on monocytes and
granulocytes

| | | | % Reduction |
|---|---|---|---|
| | | | in |
| PBS | MFI | Normalization | Monocytes |
| 605-CD20 w/o Empigen_Lot #1 | 790 | | |
| 605-CD20 with Empigen_Lot #1 | 1720.3 | 930.3 | |
| 605-CD20 w/o Empigen_Lot #2 | 978.2 | 188.2 | 80% |
| 605-CD20 with Empigen_Lot #2 | 1524.3 | 734.3 | |
| PBS | 911.3 | 121.3 | 83% |
| 786-CD103 w/o Empigen_Lot #1 | 169.9 | | |
| 786-CD103 with Empigen_Lot #1 | 706.4 | 536.5 | |
| 786-CD103 w/o Empigen_Lot #2 | 283.7 | 113.8 | 79% |
| 786-CD103 with Empigen_Lot #2 | 790.3 | 620.4 | |

% Reduction in Granulocytes
Test

| | | | % Reduction |
|---|---|---|---|
| | | | in |
| PBS | MFI | Normalization | Granulocytes |
| 605-CD20 w/o Empigen_Lot #1 | 1241.8 | | |
| 605-CD20 with Empigen_Lot #1 | 1545.3 | 303.5 | |
| 605-CD20 w/o Empigen_Lot #2 | 1358.7 | 116.9 | 61% |
| 605-CD20 with Empigen_Lot #2 | 1491.7 | 249.9 | |
| PBS | 1267.3 | 25.5 | 90% |
| 786-CD103 w/o Empigen_Lot #1 | 180.9 | | |
| 786-CD103 with Empigen_Lot #1 | 561.7 | 380.8 | |
| 786-CD103 w/o Empigen_Lot #2 | 223.9 | 43 | 89% |
| 786-CD103 with Empigen_Lot #2 | 719.9 | 539 | |

### Example 10: Effect of EMPIGEN BB® on Sample Cell Integrity

EMPIGEN BB® is a surfactant and, as such, could cause a permeabilization of cell membranes, leading to cell death. From the studies described herein, it was concluded the concentration at which EMPIGEN BB®) that is used with the polymer dye conjugate does not induce whole blood cell permeabilization or death and does not affect performance of the conjugate

The micellar concentration was studied in samples and during the staining to be sure not to exceed critical micellar concentration (CMC). See Table 5, which shows the evaluation of the CMC in the conjugate formulation and during staining. CMC was studied in the conjugate formulation and during the staining in 100 μL whole blood. Experiments were conducted to evaluate the impact of addition of EMPIGEN BB® on whole blood cell integrity and also on peripheral blood mononuclear cells (PMBCs).

TABLE 5
Empigen BB ® Concentration in Conjugate Formulation and
during Staining

| Formulation in the vial | 0.2% | | |
|---|---|---|---|
| | 2 | g/L | |
| | 279 | g/mol | |
| | 0.007168M | | |
| | 7.2 | mM | > CMC within the conjugate formulation |
| During the staining | 100 | μL WB | |
| | 10 | μl conjugate | |
| | 11 | Dilution factor | |
| | 0.65 | mM | < CMC during the staining |

The percentage of dead cells in whole blood sample with the 7-AAD was evaluated. 7-AAD is a DNA marker, the staining is positive when the cellular membrane is permeabilized. CD19-SNv428 D19-094 without EMPIGEN BB®@(negative control) and with 0.06%, 0.12%, and 0.2% EMPIGEN BB®, was tested on four donors' whole blood, with the 7-ADD, to evaluate the percentage of dead cells in each condition. The goal of the experiment was to verify if the percentage of dead cells is not increased by the EMPIGEN BB® concentration.

Two whole blood samples that had been preserved for greater than 24 hours were added as positive control of 7-AAD staining.

Protocol for 7-AAD staining:
100 μl whole blood+10 μl CD19-SNv428+/−Empigen, incubation 20 minutes.
Lyse with Versalyse, one wash,
Resuspension in 500 μl PBS 1×
Add 20 μl of 7-AAD (Ref B88526), incubation 15-20 minutes
Navios flow cytometer acquisition: FL4 for 7-AAD and FL9 for CD19-SNv428

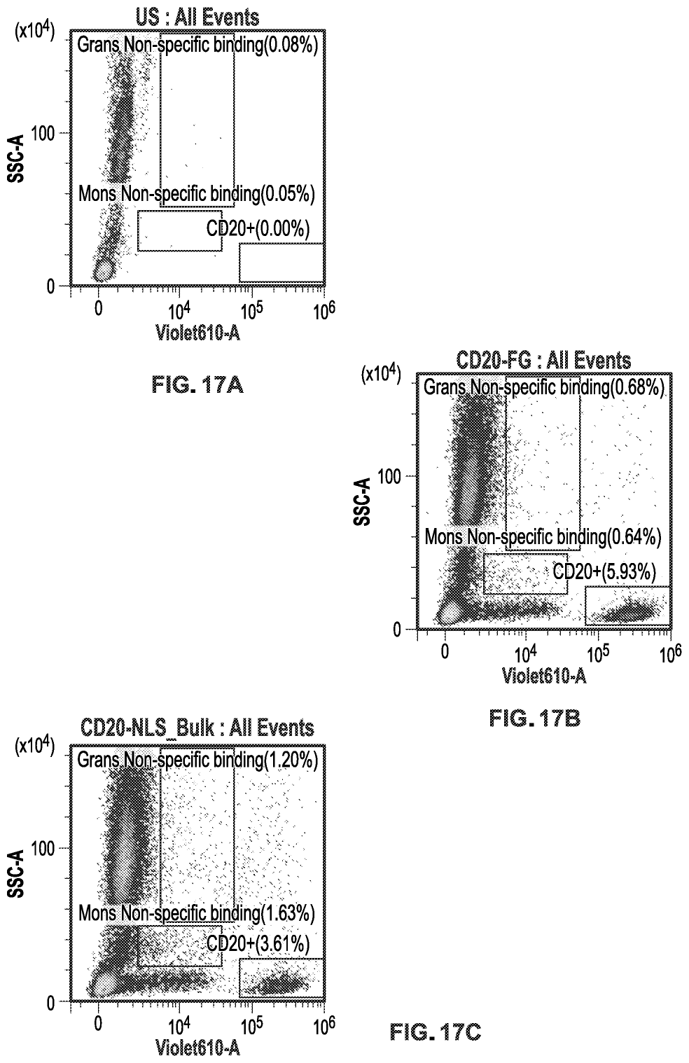

The data are presented in FIG. 16.

In conclusion, EMPIGEN BB® was proven to be effective in reducing non-specific interaction of the polymer dye conjugates described herein (SN 605-CD20, SN786-CD103, SN428-CD25, SN428-CD19 and SN428-CD22). EMPIGEN BB® efficiently reduces non-specific background binding with the monocyte and granulocyte population when tested on five specificities of conjugates. This efficiency of EMPIGEN BB® is not donor dependent. When the conjugates were compared with BV786-CD103 and BV 421, there was clear differentiation of reduction of monocyte non-specific pullout. In addition to its performance, the presence of EMPIGEN BB® with the polymer dye conjugate did not induce whole blood cell membrane permeabilization and didn't induce whole blood cell death at a concentration of up to at least 0.2% in the compositions.

### Example 11: Effect of Nonionic Surfactants

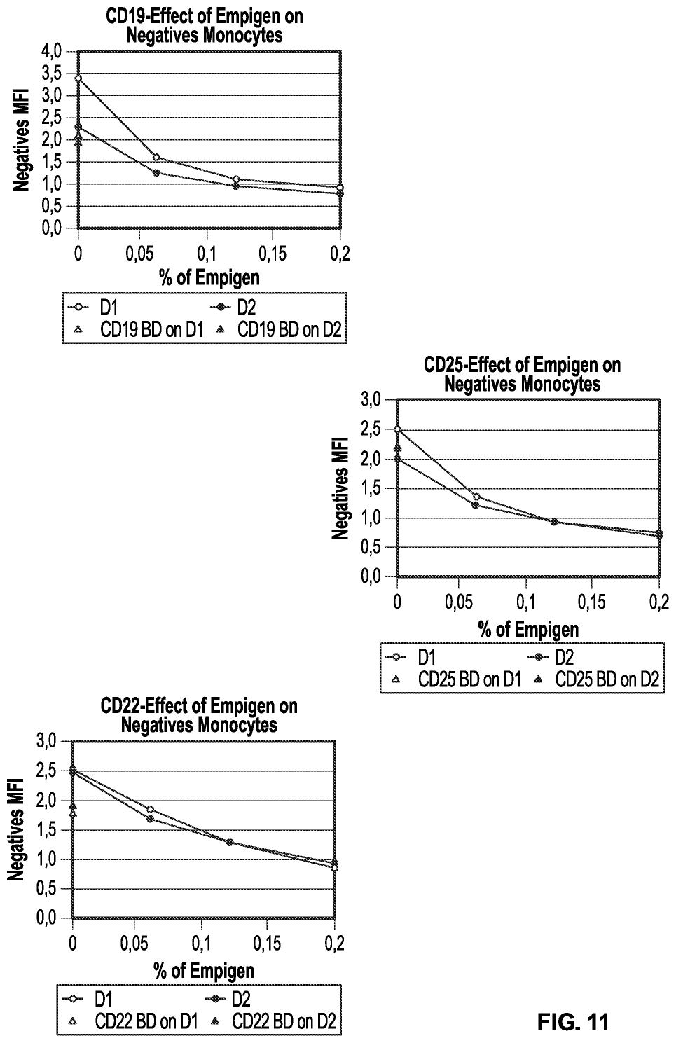

Nonionic surfactants Tween-20, tergitol, NP-40 and Pluronic F-68 (PF-68) were additional detergents/surfactants that were tested to remove non-specific binding of the conjugates described herein on monocytes. FIG. 9 shows the inefficiency of Tween-20 and PF-68 in avoiding non-specific binding of conjugates on monocytes.

### Example 12: Effect of Protein Blockers

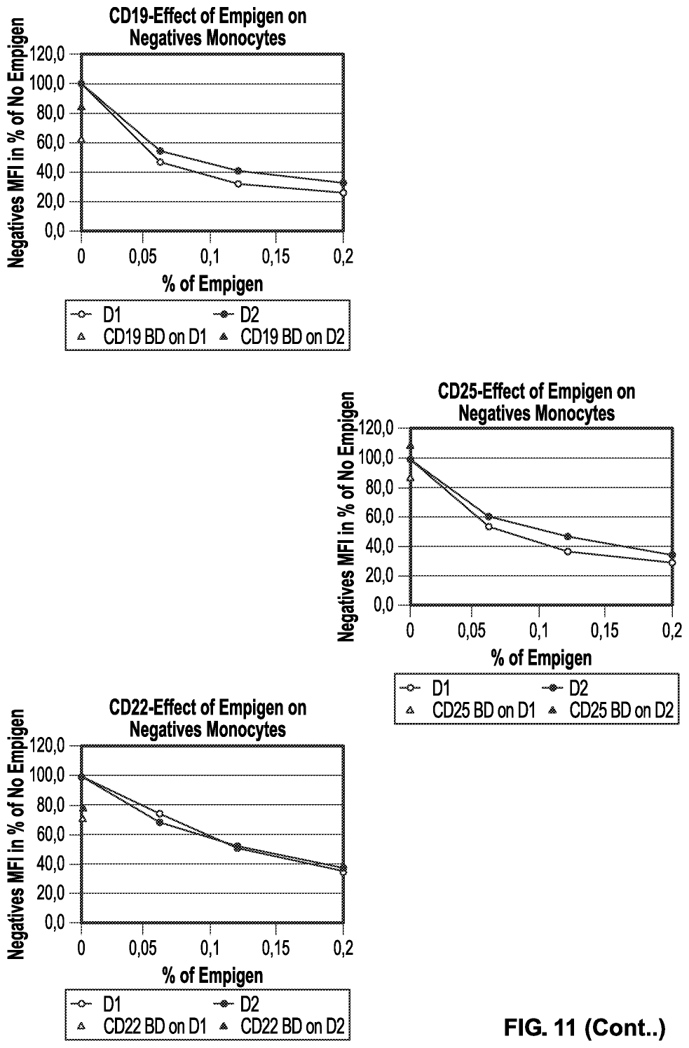

The issue of non-specific interaction on monocytes was also observed with conventional tandem dyes (e.g., PC5, PC5.5, PC7, AA700 available from Beckman Coulter, Inc.). To overcome this issue, it was thought that BSA-ox (oxidized BSA) and BSA-Cy5-ox (oxidized Cy5-BSA), which are known protein blockers, might prevent non-specific binding. But it was found that BSA, BSA-ox, and BSA-Cy5-ox were all inefficient in controlling the non-specific interactions of polymer dye conjugates with the granulocytes/monocytes. See FIG. 10.

### Example 13: Effect of Anionic Surfactant on Non-Specific Binding

Anionic surfactant N-lauryl sarcosine (NLS) was found to be effective at reducing non-specific staining on monocytes and granulocytes at 0.16% and 0.08% (w/v).

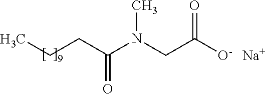

NLS is an anionic surfactant having a CMC of 14.57 mM (30° C.). NLS sodium was evaluated to determine effective concentration for preventing non-specific binding of polymer dye conjugates (SN v605-CD20) on monocytes and granulocytes.

In this example, different concentrations (0.16%, 0.08%, 0.04% and 0.02% w/v) of NLS was formulated with SN v605 CD20 conjugate in the presence of BSA and sodium azide and stained peripheral blood samples. Flow cytometry was performed following sample staining.

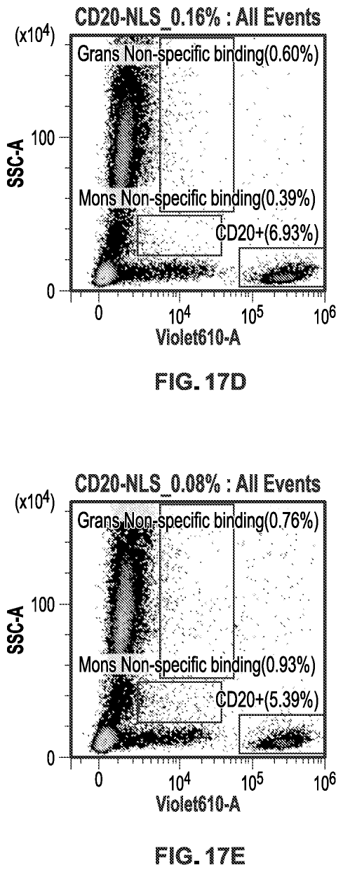

FIG. 17A-E show the dot-plots of blood samples in the absence and presence of conjugates with Empigen/without Empigen/NLS at different concentrations. A dot plot of a peripheral blood sample without single color conjugate is shown in FIG. 17A, evident as there is no population in the CD20+ gate.

A positive control dot plot of a peripheral blood sample in the presence of CD20-SN v605 single-color conjugate in a buffer composition containing BSA, sodium azide, and zwitterionic surfactant Empigen BB® as additives is shown in FIG. 17B. When compared to negative control dot plot (FIG. 17C), the % population in the gate “Mons Non-specific binding” (0.64%) and “Grans Non-specific binding” (0.68%) are each considerably reduced, indicating the effectiveness of Empigen BB® in preventing non-specific binding to monocytes and granulocytes.

A negative control dot plot of a peripheral blood sample in the presence of CD20-SN v605 single-color conjugate in a buffer composition containing only BSA and sodium azide as additives is shown in FIG. 17C. The granulocyte non-specific staining was 1.20%, and the monocyte non-specific staining was 1.63% without surfactant

A test dot plot of a peripheral blood sample in the presence of CD20-SN v605 single-color conjugate in a buffer composition containing BSA, sodium azide, and NLS (0.16% w/v) as additives is shown in FIG. 17D. Non-specific staining of granulocytes was substantially reduced to 0.60%, and monocyte non-specific staining was substantially reduced to 0.39%, compared to negative control (FIG. 17C).

A test dot plot of a peripheral blood sample in the presence of CD20-SN v605 single-color conjugate in a buffer composition containing BSA, sodium azide, and NLS (0.08% w/v) as additives is shown in FIG. 17E. Non-specific staining of granulocytes was reduced to 0.76%, and monocyte non-specific staining was reduced to 0.93%, compared to negative control (FIG. 17C).

The dot plot of FIG. 17B (Empigen BB®) when compared to FIGS. 17D and 17E (NLS) shows the % population in the gate “Mons Non-specific binding” and “Grans Non-specific binding” are very similar, indicating the effectiveness of NLS equivalent to Empigen in preventing non-specific binding on cells at 0.16% and 0.08%

The effective concentration of NLS was found to be 0.16% to 0.08% w/v to reduce or eliminate non-specific staining on monocytes and granulocytes in CD20-SN v605, therefore this concentration range of NLS anionic surfactant was demonstrated to be effective to reduce non-specific binding in single color fluorescent polymer dye conjugate compositions.

1. A method for reducing or eliminating non-specific binding of at least one polymer dye conjugate in a biological sample, the method comprising:
contacting the at least one polymer dye conjugate with at least one zwitterionic or anionic surfactant before, during, or after the polymer dye conjugate is contacted with a blood sample, the contacting resulting in decreased non-specific binding of the at least one polymer dye conjugate to a cell in the biological sample.

2. The method according to claim 1, wherein the decreased non-specific binding comprises decreased non-specific binding to a white blood cell in the biological sample; optionally wherein the biological sample is a blood sample.

3. The method according to claim 2, wherein the white blood cell is selected from the group consisting of monocytes and granulocytes.

4. The method according to claim 1, comprising contacting the surfactant with the biological sample before contacting the polymer dye conjugate with the biological sample.

5. The method according to claim 1, comprising contacting the polymer dye conjugate with the surfactant before the polymer dye conjugate and surfactant are contacted with the biological sample.

6. The method according to claim 1, wherein the surfactant is a compound of the formula:
R^{1′}[CO—X(CH₂)ⱼ]_{g}—[N⁺(R^{2′})(R^{3′})]ₖ—(CH₂)_{f}—[CH(OH)CH₂]ₕ—Y⁻, wherein
R^{1′} is a saturated or unsaturated C₅₋₂₄ alkyl;
X is NH, NR^{4′}, wherein R^{4′} is C₁₋₄ alkyl, O or S;
j is an integer from 1 to 10;
g is 0 or 1;
R^{2′} and R^{3′} are independently a C₁₋₄ alkyl;
k is 0 or 1;
the hydroxyl is optionally substituted by methyl, ethyl, hydroxymethyl, or hydroxyethyl;
f is an integer from 0 to 4;
h is 0 or 1; and
Y is COO, SO₃, OPO(OR^{5′})O, or P(O)(OR^{5′})O, wherein R^{5′} is H or C₁₋₄ alkyl.

7. The method of claim 6, wherein the surfactant is a zwitterionic surfactant compound of the formula:
R^{1′}[CO—X(CH₂)ⱼ]_{g}—N⁺(R^{2′})(R^{3′})—(CH₂)_{f}—[CH(OH)CH₂]ₕ—Y⁻,
wherein:
R^{1′} is saturated or unsaturated C₅₋₂₄ alkyl;
X is NH or NR^{4′}, wherein R^{4′} is C₁₋₄ alkyl, O or S;
j is an integer from 1 to 10;
g is 0 or 1;
R^{2′} and R^{3′} are independently a C₁₋₄ alkyl;
the hydroxyl is optionally substituted by methyl, ethyl, hydroxymethyl, or hydroxyethyl;
f is an integer from 1 to 4;
h is 0 or 1; and
Y is COO, SO₃, OPO(OR^{5′})O or P(O)(OR^{5′})O, wherein R^{5′} is H or a C₁₋₄ alkyl residue.

8. The method of claim 7, wherein the zwitterionic surfactant is a compound of the formula:
R^{1′}—N⁺(CH₃)₂—CH₂COO⁻;
R^{1′}—CO—NH(CH₂)₃—N⁺(CH₃)₂—CH₂COO⁻;
R^{1′}—N⁺(CH₃)₂—CH₂CH(OH)CH₂SO₃⁻; or
R^{1′}—CO—NH—(CH₂)₃—N⁺(CH₃)₂—CH₂CH(OH)CH₂SO₃⁻.

9. The method of claim 8, wherein the surfactant is selected from the group consisting of almondamidopropyl betaine, apricotamidopropyl betaine, avocadamidopropyl betaine, babassuamidopropyl betaine, behenamidopropyl betaine, behenyl betaine, canolamidopropyl betaine, capryl/capramidopropyl betaine, carnitine, cetyl betaine, cocamidoethyl betaine, cocamidopropyl betaine, cocamidopropyl hydroxysultaine, coco betaine, coco hydroxysultaine, coco/oleamidopropyl betaine, coco sultaine, decyl betaine, dihydroxyethyl oleyl glycinate, dihydroxyethyl soy glycinate, dihydroxyethyl stearyl glycinate, dihydroxyethyl tallow glycinate, dimethicone propyl PG-betaine, drucamidopropyl hydroxysultaine, hydrogenated tallow betaine, isostearamidopropyl betaine, lauramidopropyl betaine, lauryl betaine, lauryl hydroxysultaine, lauryl sultaine, milk amidopropyl betaine, milkamidopropyl betaine, myristamidopropyl betaine, myristyl betaine, oleamidopropyl betaine, oleamidopropyl hydroxysultaine, oleyl betaine, olivamidopropyl betaine, palmamidopropyl betaine, palmitamidopropyl betaine, palmitoyl carnitine, palm kernel amidopropyl betaine, polytetrafluoroethylene acetoxypropyl betaine, ricinoleamidopropyl betaine, sesamidopropyl betaine, soyamidopropyl betaine, stearamidopropyl betaine, stearyl betaine, tallowamidopropyl betaine, tallowamidopropyl hydroxysultaine, tallow betaine, tallow dihydroxyethyl betaine, undecylenamidopropyl betaine, and wheat germ amidopropyl betaine.

10. The method of claim 9, wherein the surfactant is lauryl betaine.

11. The method according to claim 1, wherein the surfactant is an anionic surfactant compound of the formula:
R^{1′}[CO—X(CH₂)ⱼ]_{g}—(CH₂)_{f}—[CH(OH)CH₂]ₕ—Y⁻, wherein
R^{1′} is a saturated or unsaturated C₅₋₂₄ alkyl;
X is NH, NR^{4′}, wherein R^{4′} is C₁₋₄ alkyl, O, or S;
j is an integer from 1 to 10;
g is 0 or 1;
the hydroxyl is optionally substituted by methyl, ethyl, hydroxymethyl, or hydroxyethyl;
f is an integer from 0 to 4;
h is 0 or 1; and
Y is COO, SO₃, OPO(OR^{5′})O or P(O)(OR^{5′})O, wherein R^{5′} is H or C₁₋₄ alkyl and wherein the anionic surfactant may be in acidic form, or sodium, or potassium salt forms thereof.

12. The method according to claim 11, wherein the anionic surfactant is a compound according to the formula
R^{1′}—CO—N(CH₃)—CH₂—COO⁻; or
R^{1′}—CO—N(CH₃)—CH₂—SO₃—, and the anionic surfactant is in acidic form, or sodium, or potassium salts thereof, wherein
R^{1′} is a saturated or unsaturated C₅₋₂₄ alkyl.

13. The method according to claim 12, wherein the anionic surfactant is selected from the group consisting of N-lauroyl sarcosine, sodium lauroylsarcosinate, sodium palmitoyl sarcosinate, sodium stearoyl sarcosinate, N-methyl-N-(1-oxotetradecyl)-glycine sodium salt, sodium caproyl sarcosinate, sodium capryloyl sarcosinate, N-methyl-N-(1-oxo-9-octadecen-1-yl)-glycine, sodium salt, sodium oleoyl sarcosinate, and sodium linoleoyl sarcosinate.

14. The method according to claim 13, wherein the anionic surfactant is N-lauroyl sarcosine or a salt thereof.

15. The method according to claim 1, wherein the polymer dye conjugate comprises a binding partner conjugated to a polymer dye having the structure of Formula III: 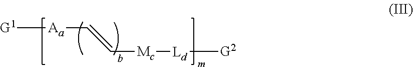
wherein,
each A is independently selected from the group consisting of an aromatic co-monomer and a heteroaromatic co-monomer;
each optional M is independently selected from the group consisting of an aromatic co-monomer, a heteroaromatic co-monomer, a bandgap-modifying monomer, optionally substituted ethylene, and ethynylene, and is evenly or randomly distributed along the polymer main chain;
each optional L is a linker moiety;
each G¹ and G² are independently selected from an unmodified polymer terminus and a modified polymer terminus;
a, c, and d define the mol % of each unit which each can be evenly or randomly repeated and where each a is a mol % from 10 to 100%, each c is a mol % from 0 to 90%, and each d is a mol % from 0 to 25%;
each b is independently 0 or 1; and
each m is an integer from 1 to about 10,000.

16. The method according to claim 15, wherein A is selected from the group consisting of a DHP moiety, a fluorene moiety, and a DHP moiety and a fluorene moiety.

17. The method according to claim 15, wherein the at least one polymer dye conjugate comprises a binding partner conjugated to a polymer having a structure according to Formula I: 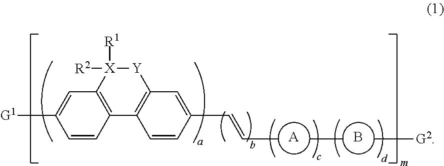
wherein
each X is independently C or Si;
each Y is independently CR¹R² or SiR¹R²;
each R¹ is independently an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety: 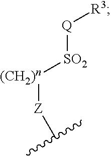
each R² is independently H, alkyl, alkenyl, alkynyl, cycloalkyl, haloalkyl, alkoxy, (hetero)aryloxy, aryl, (hetero)arylamino, a PEG group, an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety 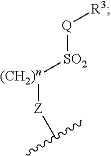
each R³ is independently selected from the group consisting of H, alkyl, alkene, alkyne, cycloalkyl, haloalkyl, alkoxy, (hetero)aryloxy, aryl, (hetero)arylamino, and a PEG group;
each Z is independently selected from the group consisting of C, O, and N;
each Q is independently selected from the group consisting of a bond, NH, NR⁴, and CH₂; and
each subscript n is independently an integer from 0 to 20;
each M is a unit capable of altering the polymer band gap and are evenly or randomly distributed along the polymer main chain;
L is a linker;
G¹ and G², which are each independently selected from the group consisting of hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted dihydrophenanthrene (DHP), optionally substituted fluorene, aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof;
a, c, and d, define the mol % of each unit within the structure which each can be evenly or randomly repeated and where a is a mol % from 10 to 100%, c is a mol % from 0 to 90%, and each d is a mol % from 0 to 25%;
each b is independently 0 or 1;
m is an integer from 1 to about 10,000; and
each n is independently an integer from 1 to 20.

18. The method according to claim 17, wherein
L is an aryl or heteroaryl group evenly or randomly distributed along the polymer main chain and substituted with one or more pendant chains terminated with a functional group selected from the group consisting of amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof conjugated to a binding partner.

19. The method according to claim 15, wherein the binding partner is a molecule or complex of molecules capable of specifically binding to target analyte.

20. The method according to claim 19, wherein the binding partner is a protein, an affinity ligand, an antibody, or an antibody fragment.

21. The method according to claim 20, wherein the binding partner is selected from the group consisting of a monoclonal antibody, a polyclonal antibody, an immunoglobulin, an immunologically active portion of an immunoglobulin, a single chain antibody, Fab fragment, Fab′ fragment, and F(ab′)2 fragments, and scFv fragment.

22. A composition comprising
a polymer dye conjugate;
an aqueous buffer; and
a zwitterionic or anionic surfactant.

23. The composition according to claim 22, further comprising a non-polymeric dye conjugate.

24. The composition according to claim 22, wherein the zwitterionic or anionic surfactant is at a concentration below the critical micellar concentration (CMC).

25. The composition according to claim 24, wherein the surfactant is at a concentration of 0.05 to 0.25% (w/v), 0.06 to 0.20% (w/v), or 0.08 to 0.16% (w/v).

26. The composition according to claim 22, wherein the aqueous buffer comprises one or more additional additive(s) selected from the group consisting of a protein stabilizer, a preservative, and an additional surfactant.

27. The composition according to claim 22, wherein following contacting the composition with a biological sample, the polymer dye conjugate exhibits reduced non-specific binding to cells in the sample as compared to non-specific binding of the polymer dye conjugate to cells in the sample when the polymer dye conjugate is contacted with the sample without zwitterionic or anionic surfactant.

28. The composition according to claim 27, wherein the sample is a blood sample and the cells are white blood cells selected from the group consisting of monocytes and granulocytes.

29. The composition according to claim 22, wherein the surfactant is a compound of the formula:
R^{1′}[CO—X(CH₂)ⱼ]_{g}—[N⁺(R^{2′})(R^{3′})]ₖ—(CH₂)_{f}—[CH(OH)CH₂]ₕ—Y⁻, wherein
R^{1′} is a saturated or unsaturated C₅₋₂₄ alkyl;
X is NH, NR^{4′}, wherein R^{4′} is C₁₋₄ alkyl, O or S;
j is an integer from 1 to 10;
g is 0 or 1;
R^{2′} and R^{3′} are independently a C₁₋₄ alkyl;
k is 0 or 1;
the hydroxyl is optionally substituted by methyl, ethyl, hydroxymethyl, or hydroxyethyl;
f is an integer from 0 to 4;
h is 0 or 1; and
Y is COO, SO₃, OPO(OR^{5′})O, or P(O)(OR^{5′})O, wherein R^{5′} is H or C₁₋₄ alkyl, and when k=0, the surfactant may be in acidic form, or sodium, or potassium salts thereof.

30. The composition according to claim 22, wherein the polymer dye conjugate comprises a binding partner conjugated to a polymer dye having the structure of Formula III: 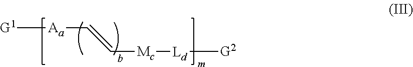
wherein
each A is independently selected from the group consisting of an aromatic co-monomer and a heteroaromatic co-monomer;
each optional M is independently selected from the group consisting of an aromatic co-monomer, a heteroaromatic co-monomer, a bandgap-modifying monomer, optionally substituted ethylene, and ethynylene, and is evenly or randomly distributed along the polymer main chain;
each optional L is a linker moiety;
each G¹ and G² are independently selected from an unmodified polymer terminus and a modified polymer terminus;
a, c, and d define the mol % of each unit which each can be evenly or randomly repeated and where each a is a mol % from 10 to 100%, each c is a mol % from 0 to 90%, and each d is a mol % from 0 to 25%;
each b is independently 0 or 1;
and each m is an integer from 1 to about 10,000.

31. The composition according to claim 30,
where the polymer dye conjugate comprises a binding partner conjugated to a polymer dye having a structure according to Formula I: 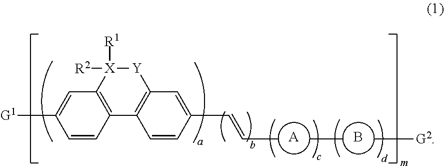
wherein
each X is independently C or Si;
each Y is independently CR¹R² or SiR¹R²;
each R¹ is independently an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety: 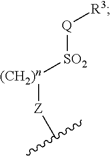
each R² is independently H, alkyl, alkenyl, alkynyl, cycloalkyl, haloalkyl, alkoxy, (hetero)aryloxy, aryl, (hetero)arylamino, a PEG group, an ammonium alkyl salt, an ammonium alkyloxy salt, an ammonium oligoether salt, a sulfonate alkyl salt, a sulfonate alkoxy salt, a sulfonate oligoether salt, a sulfonamido oligoether, or a moiety 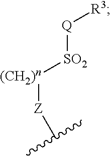
each R³ is independently selected from the group consisting of H, alkyl, alkene, alkyne, cycloalkyl, haloalkyl, alkoxy, (hetero)aryloxy, aryl, (hetero)arylamino, and a PEG group;
each Z is independently selected from the group consisting of C, O, and N;
each Q is independently selected from the group consisting of a bond, NH, NR⁴, and CH₂; and
each subscript n is independently an integer from 0 to 20;
each M is a unit capable of altering the polymer band gap and are evenly or randomly distributed along the polymer main chain;
L is a linker;
G¹ and G², which are each independently selected from the group consisting of hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted dihydrophenanthrene (DHP), optionally substituted fluorene, aryl or heteroaryl substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof;
a, c, and d, define the mol % of each unit within the structure which each can be evenly or randomly repeated and where a is a mol % from 10 to 100%, c is a mol % from 0 to 90%, and each d is a mol % from 0 to 25%;
each b is independently 0 or 1;
m is an integer from 1 to about 10,000; and
each n is independently an integer from 1 to 20.
